(19) United States
(12) Patent Application Publication
(10) Pub. No.: US 2014/0350380 A1
(43) Pub. Date: Nov. 27, 2014

(54) IDENTIFICATION AND USES OF BRAIN ACTIVITY NETWORKS

(71) Applicant: THE FEINSTEIN INSTITUTE FOR MEDICAL RESEARCH, Manhasset, NY (US)

(72) Inventor: David Eidelberg, New York, NY (US)

(73) Assignee: THE FEINSTEIN INSTITUTE FOR MEDICAL RESEARCH, Manhasset, NY (US)

(21) Appl. No.: 14/345,331

(22) PCT Filed: Sep. 18, 2012
(86) PCT No.: PCT/US12/55913

§ 371 Date: Mar. 17, 2014

Related U.S. Application Data
(60) Provisional application No. 61/536,351, filed on Sep. 19, 2011.

(51) Int. Cl. G06T 7/00; A61B 6/03; A61B 6/00; A61B 5/055

(52) U.S. Cl. CPC G06T 7/0012; A61B 5/055; A61B 6/037; A61B 6/501; G06T 2207/10088; G06T 2207/10104; G06T 2207/30016
USPC 600/410; 600/436

(57) ABSTRACT

Methods for identifying networks correlating with placebo effects, progression of neurological disease symptoms, progression of pre-phenoconversion states of neurological diseases, and efficacious/non-efficacious candidate treatments for neurological diseases are provided.

### CROSS-REFERENCE TO RELATED APPLICATIONS

This application claims benefit of U.S. Provisional Application No. 61/536,351, filed Sep. 19, 2011, the contents of which are hereby incorporated by reference.

### STATEMENT OF GOVERNMENT SUPPORT

This invention was made with government support under grant numbers R01 NS 37564, R01 NS 35069 and R01 NS 40068 awarded by the National Institutes of Health; grant number R01 MH 01579 awarded by the National Institutes of Mental Health; and grant number M01 RR 018535 awarded by the National Center for Research Resources. The government has certain rights in the invention.

### BACKGROUND OF THE INVENTION

Throughout this application various patents and other publications are referred to in parenthesis. Full citations for the references may be found at the end of the specification. The disclosures of these patents and publications are hereby incorporated by reference in their entirety into the subject application to more fully describe the art to which the subject invention pertains.

Spatial patterns for the diagnosis of brain disease have previously been developed (e.g. see U.S. Pat. Nos. 5,632,279 and 5,873,823). These relate to diagnostic brain patterns derived from populations of patients and controls. These methods are limited however in their relative insensitivity to disease progression and inability to identify treatment-specific (as opposed to disease-specific) network changes.

The present invention address the need for methods to assess disease progression and pre-phenoconversion states and provides the ability to monitor treatment-specific network changes.

### SUMMARY OF THE INVENTION

A method is provided for identifying a pattern of brain activity associated with a placebo effect response to a placebo treatment for a disease or disorder comprising: determining, by positron emission tomography or magnetic resonance imaging (MRI) in a subject receiving, or who has received, the placebo treatment for the disease or disorder, functional activity at each of a plurality of coordinates of the subject's brain during at least two different time points and identifying, through spatial co-variance analysis of the functional activity, which coordinates show a consistent trend over the at least two different time points in functional activity correlating with a placebo effect response to the placebo treatment, so as to thereby determine a pattern of brain activity associated with a placebo effect response to a placebo treatment for a disease or disorder.

A method is provided for determining efficacy of a candidate treatment, administered to a subject having a neurological or psychological disease, on a rate of progression of the disease comprising:

a) determining, by positron emission tomography or functional magnetic resonance imaging (fMRI) during administration of or after administration of the candidate treatment to the subject, functional activity at each of a plurality of predetermined coordinates of the subject's brain so as to determine a first pattern of activity, which coordinates have previously been identified through spatial co-variance analysis of functional activity as determined by positron emission tomography or fMRI in the brain of the subject or in the brain(s) of one or more other subjects suffering from the neurological or psychological disease during at least two different time points while the subject was, or subjects were, exhibiting the symptom or disease as showing a consistent trend in functional activity which correlates with worsening of the disease; and
b) comparing the first pattern of activity determined in step a) with a previously determined baseline pattern of activity,
wherein an expression of the first pattern of activity lower than the previously determined baseline pattern of activity indicates that the candidate treatment is efficacious in reducing the rate of progression of the disease, and wherein an expression of the first pattern of activity higher than the previously determined baseline pattern of activity indicates that the candidate treatment is not efficacious in reducing the rate of progression of the disease.

A method is also provided for identifying a pattern of brain activity specifically associated with a symptom of a neurological or psychological disease comprising:

determining, by positron emission tomography or functional magnetic resonance imaging (fMRI) in a subject exhibiting the symptom, functional activity at each of a plurality of coordinates of the subject's brain during at least two different time points and identifying, through spatial co-variance analysis of the functional activity, which coordinates show a consistent trend over the at least two different time points in functional activity correlating with the symptom,
so as to determine a baseline pattern of activity specifically associated with the symptom of the neurological or psychological disease.

A method is also provided for identifying a pattern of brain activity specifically associated with worsening of a symptom of a neurological or psychological disease comprising:

determining, by positron emission tomography or functional magnetic resonance imaging (fMRI) in a subject exhibiting the symptom, functional activity at each of a plurality of coordinates of the subject's brain during at least two different time points and identifying, through spatial co-variance analysis of the functional activity, which coordinates show a consistent trend over the at least two different time points in functional activity correlating with the worsening of the symptom,
so as to thereby determine a baseline pattern of activity specifically associated with the worsening of symptom of the neurological or psychological disease.

A method is also provided for identifying a pattern of brain activity specifically associated with efficacious treatment of a symptom of a neurological or psychological disease comprising:

determining, by positron emission tomography or functional magnetic resonance imaging (fMRI) in a subject exhibiting the symptom and being treated with a treatment efficacious for that symptom, functional activity at each of a plurality of coordinates of the subject's brain during at least two different time points and identifying, through spatial co-variance analysis of the functional activity, which coordinates show a consistent trend over the at least two different time points in functional activity correlating with efficacious treatment of the symptom,
so as to thereby determine a baseline pattern of activity specifically associated with efficacious treatment of the symptom of the neurological or psychological disease.

A method is also provided for identifying a pattern of brain activity specifically associated with a pre-phenoconversion state of a neurological disease comprising:

determining, by positron emission tomography or functional magnetic resonance imaging (fMRI) in a subject in a pre-phenoconversion state, functional activity at each of a plurality of coordinates of the subject's brain during at least two different time points and identifying, through spatial co-variance analysis of the functional activity, which coordinates show a consistent trend in functional activity over the at least two different time points correlating with the pre-phenoconversion state,
so as to thereby determine a pattern of brain activity specifically associated with the pre-phenoconversion state of the neurological disease.

A method is also provided for identifying a pattern of brain activity specifically associated with predisposition to a neurological disease comprising:

determining, by positron emission tomography or functional magnetic resonance imaging (fMRI) in a subject predisposed to the neurological disease, functional activity at each of a plurality of coordinates of the subject's brain during at least two different time points and identifying, through spatial co-variance analysis of the functional activity, which coordinates show a consistent trend in functional activity over the at least two different time points correlating with predisposition to the neurological disease,
so as to thereby determine a pattern of brain activity specifically associated with predisposition to a neurological disease.

A method is also provided of determining a pre-phenoconversion subject as likely to phenoconvert to a neurological disease within a predetermined time period comprising determining, by positron emission tomography or functional magnetic resonance imaging (fMRI), functional activity at each of a plurality of predetermined coordinates of the pre-phenoconversion subject's brain so as to determine a first pattern of activity, and comparing the first pattern of activity to a baseline pattern of activity which correlates with a pre-phenoconversion state and does not correlate with a phenoconversion state,

wherein an expression of the first pattern of activity in excess of a predetermined multiple of the baseline pattern of activity indicates that the subject is likely to phenoconvert to the neurological disease within the predetermined time period, and wherein an expression of the first pattern of activity lower than a predetermined multiple of the baseline pattern of activity indicates that the subject is not likely to phenoconvert to the neurological disease within the predetermined time period.

A method is also provided for identifying a pattern of brain activity specifically associated with a placebo effect response to a placebo treatment for a disease or disorder comprising:

determining, by positron emission tomography or functional magnetic resonance imaging (fMRI) in a subject receiving or who has received the placebo treatment for the disease or disorder functional activity at each of a plurality of coordinates of the subject's brain during at least two different time points and identifying, through spatial co-variance analysis of the functional activity, which coordinates show a consistent trend over the at least two different time points in functional activity correlating with a placebo effect response to the placebo treatment,
so as to thereby determine a pattern of brain activity specifically associated with a placebo effect response to a placebo treatment for a disease or disorder.

A system is provided for identifying related proteins, comprising: one or more data processing apparatus; and

a computer-readable medium coupled to the one or more data processing apparatus having instructions stored thereon which, when executed by the one or more data processing apparatus, cause the one or more data processing apparatus to perform one of any of the above-described methods.

A computer-readable medium is provided comprising instructions stored thereon which, when executed by a data processing apparatus, causes the data processing apparatus to perform a method of one of any of the above-described methods.

### BRIEF DESCRIPTION OF THE DRAWINGS

FIG. 1A-1C. Parkinson's disease tremor-related pattern (PDTP). (A) Spatial covariance pattern identified by ordinal trends canonical variate analysis (OrT/CVA) of FDG PET data from nine tremor dominant PD patients scanned on and off Vim stimulation (see text). The pattern was characterized by increased metabolic activity in the primary motor cortex, anterior cerebellum/dorsal pons, and the caudate/putamen. [The covariance map was overlaid on T1-weighted MR-template images. Voxel weights were thresholded at Z=2.70, p<0.01. The display represents regions that were demonstrated to be reliable (p<0.05) by bootstrap resampling]. (B) The expression of this PD tremor-related metabolic pattern (PDTP) was reduced by Vim stimulation in 10 of the 11 treated hemispheres (p<0.005, permutation test). (C) Baseline PDTP expression (i.e., off-stimulation pattern scores) correlated (r=0.85, p<0.02) with concurrent accelerometric measurements of tremor amplitude (see text).

FIG. 2. Comparison of PDTP and PDRP spatial topographies. Display of brain areas contributing to the PDTP (dark gray) and PDRP (light gray) metabolic networks. Areas of overlap between the two patterns were evident in the cerebellum, pons, and putamen. For each pattern, the voxel displays were thresholded at Z=2.70, p<0.01 and superimposed on a standard magnetic resonance imaging template.

FIG. 3A-3C. Validation of PDTP expression as a network correlate of parkinsonian tremor. (A) Bar graph showing mean PDTP (±SE) in a prospective group of 41 PD patients (black bars) and 20 age-matched healthy control subjects (white bars). The expression of this disease-related pattern was elevated in this testing group (p<0.001, relative to controls). (B) PDTP expression correlated (r=0.54, p<0.001) with UPDRS subscale ratings for tremor in the PD group. (C) The correlation of PDTP scores with tremor was significantly greater in magnitude (p<0.01; multiple regression analysis) than with subscale ratings for akinesia-rigidity (see text). (D) Bar graph showing mean PDTP subject scores (±SE) in tremor dominant and akinesia-rigidity dominant PD patients (arPD and tPD, respectively), and in normal control (NC) subjects undergoing perfusion imaging with ECD SPECT (see text). PDTP expression was significantly higher in the tPD patients than in the arPD (p<0.05) and NC groups (p=0.001).

FIG. 4A-4B. Changes in PDTP expression with disease progression. (A) Mean (±SE) off-state total motor UPDRS ratings (diamonds), and akinesia-rigidity (triangles) and tremor subscale ratings (squares), from a longitudinal cohort of early stage PD patients (n=15) followed at baseline, 24, and 48 months (Huang et al., 2007b C. Huang, C. Tang, A. Feigin, M. Lesser, Y. Ma, M. Pourfar, V. Dhawan and D. Eidelberg, Changes in network activity with the progression of Parkinson's disease. Brain, 130 (2007), pp. 1834-1846. Huang et al., 2007b). These ratings worsened over time (total motor UPDRS, p<0.0001; akinesia-rigidity: p<0.05; tremor: p=0.01; one-way RMANOVA), but at different progression rates (see text). Relative to baseline, significant increases in the akinesia-rigidity and tremor ratings (p<0.05; post-hoc Bonferroni test) were evident only at the 48-month time point. *p<0.05, ***p<0.0001, post-hoc Bonferroni test relative to baseline. (B) Mean (±SE) PDTP (squares) and PDRP (diamonds) scores at baseline, 24 and 48 months. The expression of both patterns increased significantly over time (PDTP: p=0.01; PDRP: p<0.0001; one-way RMANOVA). The time course of network activity differed for the two patterns (p<0.01), with a slower rate of progression for PDTP (0.10 point/year, p<0.05) relative to PDRP (0.51 point/year, p<0.0001). Relative to baseline, there was no change in PDTP expression at 24 months (p=0.99; post-hoc Bonferroni test), although a significant increase was evident at 48 months (p<0.05). However, significant increases in PDRP expression were evident at both the second (p<0.05) and third time points (p<0.0001). *p<0.05, ***p<0.001, post-hoc Bonferroni tests with respect to baseline.

FIG. 5A-5C. Changes in metabolic network activity with deep brain stimulation for PD tremor. (A) Bar graphs showing mean baseline PDTP expression (±SE) in the Vim DBS patients (black), the STN DBS patients (gray), and the healthy control subjects (white). There was a significant difference in PDTP expression across the three groups (p<0.001; one-way ANOVA), with comparable elevations in baseline pattern expression in both the Vim DBS (p<0.005) and STN DBS groups (p<0.001) relative to controls. (B) Baseline PDRP expression also differed across the three groups (p<0.001), with higher expression in both treatment groups relative to controls (p<0.001). Nonetheless, PDRP expression was higher in the STN than in the Vim DBS group (p<0.01). (C) Treatment-mediated changes (ON−OFF) in mean PDTP expression (±SE) in the Vim DBS patients (black), the STN DBS patients (gray), and the test-retest PD control subjects (white). Changes in PDTP expression were different across the three groups (p<0.001; one-way ANOVA), with stimulation-mediated declines in network activity in both DBS groups (Vim: p<0.001; STN: p=0.01, relative to the test-retest control group). PDTP modulation was greater with Vim than STN stimulation (p<0.05). (D) There was also a significant group difference in treatment-mediated PDRP modulation (p=0.02). Treatment-mediated reductions in PDRP expression reached significance (p<0.05) with STN stimulation, but not with Vim stimulation (p=0.16).

FIG. 6A-6B. Huntington's disease progression pattern A. Spatial covariance pattern identified by network analysis of the metabolic images from 12 premanifest HD mutation carriers (HD1) scanned at baseline, 1.5 and 4 years. The pattern topography (Table 4) was characterized by declining metabolic activity (darker areas) in the caudate/anterior putamen, mediodorsal (MD) thalamus, insula and posterior cingulate area, and in prefrontal and occipital cortex. These changes were associated with increasing metabolic activity (darkest areas) in the cerebellum, pons, and orbitofrontal cortex. The pattern was displayed as a reliability map of the voxel weights (regional loadings) on the topographic pattern based upon bootstrap resampling (1,000 iterations). The larger the absolute value of the inverse coefficient of variation (|ICV|), the smaller the variability of the voxel weight about its point estimate value. This map was thresholded at 2.33, which corresponds to p<0.01 (one-tailed). B. Pattern expression values for the longitudinal cohort of HD mutation carriers at baseline, 1.5, and 4 years. All 12 premanifest HD subjects exhibited a monotonic increase in pattern expression over this time period. Black lines denote the premanifest subjects who subsequently phenoconverted, i.e., received a clinical diagnosis of definite HD during at a later time point. Post-phenoconversion values for these subjects are represented by filled symbols. Dark gray lines denote their counterparts who did not phenoconvert, i.e., remained clinically premanifest over the course of the study. The horizontal broken line represents the mean (zero) for the original healthy control group; the dotted lines represent 2 SD above and below the normal mean.

FIG. 7A-7B. Validation of network activity in testing populations A. Prospectively computed pattern expression values for the five premanifest (open squares) and four symptomatic (filled squares) members of the original longitudinal cohort of HD mutation carriers (HD1) who were scanned at the fourth time point (7 years), and for members of an independent prospective testing group (HD2) comprised of five early symptomatic HD patients (filled triangles) and nine premanifest mutation carriers (open triangles) who participated in repeat metabolic imaging studies to assess the test-rest reproducibility of the network measurements. Subject scores (open circles) were also computed in 12 healthy control subjects (HC1); the mean and standard deviation of these values were used to standardize the corresponding network measures computed prospective in the gene carriers. A second healthy control group (HC2) was comprised of 20 subsequent normal volunteer subjects (open circles). Network values computed prospectively in these individuals were used to demonstrate the absence of significant elevations in pattern expression in gene-negative subjects. The horizontal broken line represents the mean for the HC1 group; the dotted lines represent 2 SD above and below the normal mean. B. Test-retest reproducibility was excellent (ICC=0.96, p<0.001) for the network values computed prospectively in the nine premanifest HD2 subjects who underwent repeat metabolic imaging at three weeks (see Methods). The line of identity (dotted line) falls within the 95% confidence interval of the test-retest regression line. Data from the four PET imaging laboratories that participated in the test-retest study are signified by color code.

FIG. 8A-8B. Rate of network progression in early HD A. Pattern expression values in the original longitudinal premanifest HD cohort (n=12) exhibited a linear increase with “disease time,” defined as the number of years remaining to the estimated time of clinical onset (see text), at a rate of 0.21/year (p<0.0001, individual growth model (IGM)). Black lines denote premanifest subjects who phenoconverted over the course of the study; post-phenoconversion values are depicted by filled symbols. Dark gray lines denote their counterparts who did not phenoconvert. B. In an independent longitudinal testing cohort of premanifest mutation carriers (n=21), pattern expression exhibited a similar linear increase with disease progression at a rate of 0.19/year (p<0.0005). In both A and B, the solid line represents the best fit according to the IGM; the broken curves represent the 95% confidence interval of the fit line. The horizontal broken line represents the normal mean (zero); the dotted lines represent 2 SD above and below the normal mean.

FIG. 9A-9B. Longitudinal changes in striatal D₂ receptor binding and tissue volume. A. Caudate (left) and putamen (right) D₂ receptor binding values measured using [¹¹C] raclopride and PET exhibited a linear decrease with years-to-onset (−2.1% and −1.8% of normal mean per year; p<0.005, individual growth model). The decline in the caudate was faster than for the putamen (p<0.02). B. Caudate (left) and putamen (right) tissue volume measurements acquired with MRI exhibited a linear decrease with advancing disease (−2.3% and −1.7% of the normal mean per year, p<0.0001), with similar rates of progression for both striatal subregions (p=0.27). In each plot, individual values are represented as percent of the mean (broken line) for an age-matched group of healthy control subjects; the dotted lines represent 2 SD above and below the normal mean. For the longitudinal premanifest HD cohort, data from the phenoconverters and non-phenoconverters are presented by red and blue lines, respectively. Measurements obtained before and after phenoconversion are represented by open and filled symbols. The solid line depicts the best fit of the longitudinal data according to the individual growth model; the broken curves represent the 95% confidence interval of the fit line. Caudate and putamen values for the five symptomatic subjects in the HD2 group (triangles) are provided for reference.

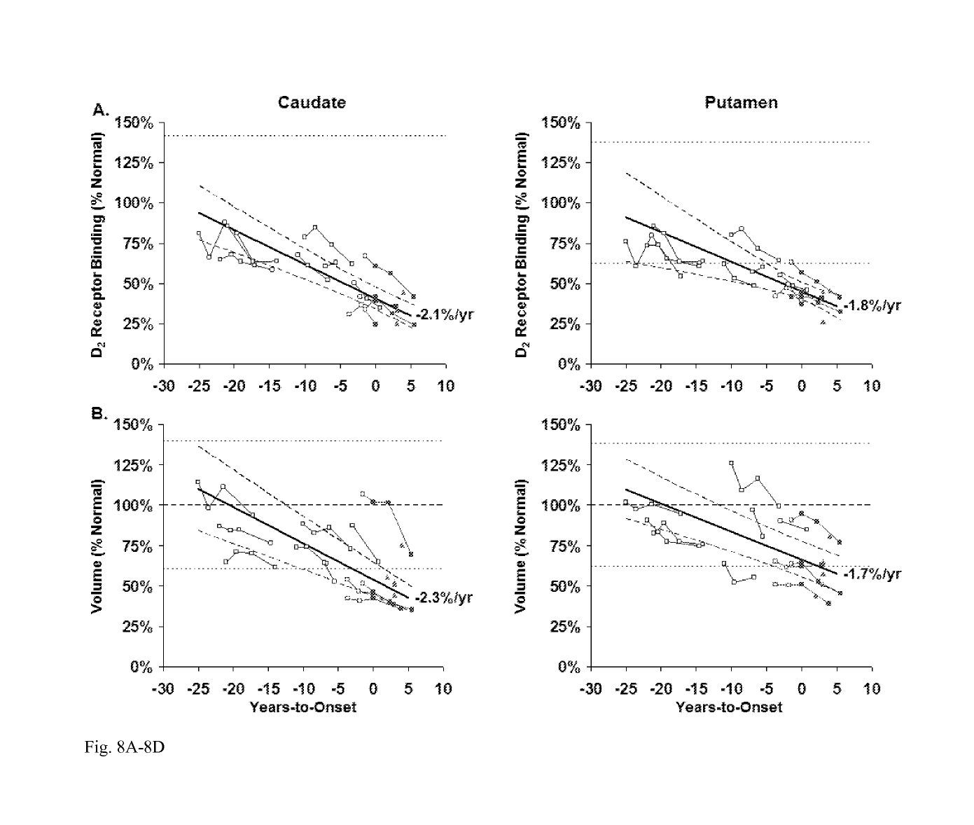

FIG. 10. Time course of disease progression: caudate D₂ receptor binding and tissue volume vs. network activity. Solid lines represent the linear trajectories for the longitudinal data according to the best fitting individual growth model. The time course of the caudate D₂ receptor binding (light gray) and tissue volume (black) measurements is displayed relative to that for the expression of the HD progression network (dark gray). There was a significant difference (interaction effect: p<0.0001) in the time course of these three measures in the longitudinal premanifest cohort. The rate of increase in pattern expression (0.21/year) was significantly greater than the rates of decline measured for caudate D₂ receptor binding (|-0.10|/year; p<0.0001) and volume (|-0.11|/year, p<0.0005). To allow for the direct comparison of network progression (increasing time course) with corresponding changes in caudate D₂ receptor binding and tissue volume (decreasing time course), the latter values were flipped and analyzed as increasing mirror lines. For each fit line, Y-axis represents the standard z-scale. Horizontal dotted line represents the normal mean values for each parameter (zero). Vertical dotted line represents the time of clinical diagnosis (when years-to-onset=0). The table (inset) gives the estimated slope (the rate of change/year) and intercept (value at the time of clinical diagnosis), 95% confidence intervals, and p-values based on the best fitting individual growth models.

FIGS. 11A-11D. Longitudinal metabolic changes in the HD progression network: regional analysis. A, B. In the longitudinal HD1 premanifest cohort, progressive declines in regional metabolic activity (p<0.0001; individual growth model) were present in (A) the caudate nucleus and anterior putamen, and (B) the mediodorsal thalamus. In these regions, metabolic activity was lower in the phenoconverters at all four time points. C, D. Regional metabolic activity concurrently increased in (C) the cerebellum (p<0.05) and (D) pons (p<0.01), Higher values were evident in the phenoconverters at all time points. Mean metabolic activity (±1 SE) for each region was displayed for the 12 longitudinal premanifest HD1 carriers (black line) at each time point. Mean progression in the phenoconverters (n=4) and the non-phenoconverters (n=8) was depicted by darkest gray and gray lines, respectively. The broken line represents mean metabolic activity for the HC1 healthy control group (n=12); the dotted lines represent 1 SE above and below the normal mean.

FIGS. 12A-12D. Effects of volume loss on the rate of network progression A. Brain regions with significant loss of tissue volume over time displayed as the statistical parametric map (SPM) of the voxel-based morphometric (VBM) scans acquired in the HD1 premanifest cohort at baseline, 1.5, and 4 years. This analysis revealed significant progression-related declines in tissue volume involving the caudate nucleus, insula, parahippocampal gyms, and prefrontal, somatosensory, precuneus and lateral occipital cortical regions. The volume loss map was displayed at a voxel level threshold of Z=3.55, p=0.001, with a false discovery rate (FDR) correction at p<0.05. B., C. HD progression pattern expression inside (B) and outside (C) the volume loss mask plotted with respect to predicted years-to-onset. In the longitudinal premanifest HD cohort, pattern expression values within the mask exhibited a linear increase with advancing disease at a rate of 0.10/year (p<0.005, individual growth model (IGM)). Outside the mask, pattern expression increased at a rate of 0.22/year (p<0.0001). For each subspace, the solid line represents the trajectory of the best fitting model; the broken curves represent the 95% confidence interval of the fit line. D. There was a significant difference (interaction effect: p<0.05, IGM) in the rates of network progression measured for the whole brain (dark gray) and those measured inside (gray) and outside (darkest gray) the volume loss mask. The rate of network progression outside the mask (0.22/year) was similar to that measured for the whole brain (0.21/year, p=0.97). These progression rates were significantly faster than that measured inside the volume loss mask (0.10/yr; p<0.01). In each plot, the broken line represents the mean value of the HC 1 healthy control group; the dotted lines represent 2 SD above and below the normal mean value.

FIG. 13. OrT/CVA derived placebo-related pattern (PlcRP). The Akaike information criterion was smallest with the linear combination of PC3 and PC4. A. For visualization, the image is z-score transformed based on all voxels in the grey matter brain mask. Several hyperactive regions were identified including subgenual anterior cingulate cortex, cerebellar vermis, inferior temporal cortex, hippocampus and amygdala. Hypoactive regions include inferior temporal, parahippocampal gyms and cuneus. The image is filtered with |ICV|>1.64 (p<0.05, one-tailed) and cluster size >100 to only show the voxels with significant reliability. B. No exception was identified in the ordinal trend, i.e., all patients subject score was increased at 6 month vs baseline (before surgery) in all improved patients (both derivation and testing group). A subset of non-improved patients' subject scores were increased (4 out of 7). Difference between improved and non-improved patients' subject scores were significant within the testing group (t(13)=2.413, p=0.031).

FIG. 14. Correlation between changes in PlcRP and changes in UPDRS motor ratings. Significant negative correlation was observed within the derivation group (r=−0.774, p=0.024) and improved patients in testing group (r=−0.780, p=0.022). No significant correlation was observed in the patients whose UPDRS motor rating was not changed or worsened (r=−0.211, p=0.650).

### DETAILED DESCRIPTION OF THE INVENTION

### Abbreviations

AIC—Akaike information criterion
DBS—deep brain stimulation (DBS)

### FDG—¹⁸F-fluorodeoxyglucose (FDG)

HD—Huntingdon's disease
HDPP—Huntingdon's disease progression pattern
MRI—magnetic resonance imaging

### OrT/CVA—Ordinal Trends Canonical Variates Analysis

PC—principal component
PCA—principal component analysis
PD—Parkinson's disease
PDRP—PD-related metabolic covariance pattern (PDRP)
PDTP—PD tremor-related metabolic pattern (PDTP)
PET—positron emission tomography (PET)
RMANOVA—one-way repeated measure analysis of variance

### UPDRS—Unified Parkinson's Disease Rating Scale (UPDRS)

Vim—ventral intermediate (Vim)

As used herein, a “candidate treatment” is any treatment or therapy, including in non-limiting examples a candidate drug, dosing regimen, dosage form, or administration technique, and which is selected for testing as to its efficacy in treating or ameliorating a disease, disorder or symptom.

As used herein, “progression” of a disease means the development, enhancement or worsening of one or more hallmarks or symptoms of the disease.

As used herein, a “pattern of activity” is constituted by brain activity (e.g. determined as metabolic activity in the brain) determined at a plurality of discrete co-ordinates in a brain of the relevant subject. A “baseline” pattern activity is one determined from, and/or selected as, a suitable baseline or control, e.g. from or in a subject not having the relevant disease, not exhibiting a symptom of the relevant disease, having a predisposition and not yet having the disease or being in a prephenoconversion state. Thus, the baseline provides a reference pattern against which expression of the pattern determined by the method can be compared for concluding the relative state or position of the tested subject.

As used herein, a “placebo effect” is the art-recognized phenomenon whereby a patient's symptoms can be alleviated by a sham treatment. A placebo effect can be seen in patients receiving a sham or simulated medical intervention.

As used herein, “predisposition” to a disease or a disorder is a state in which a subject is susceptible to developing the disease or a disorder. The susceptibility to the disease may be genetic, or extant through lifestyle, behavior and such. Such susceptibilities are known in the art and are often identified in a subject by, in the absence of genetic information, the subject exhibiting one or more risk factors for the disease or disorder.

As used herein, “correlating” with a defined state or position means showing a positive or negative correlation in direction, quantity, change in direction and/or change in quantity, with the defined state or position.

As used herein, “expression” of a pattern is the degree of exhibition of the pattern, for example quantified in units of activity or a surrogate therefor, or measured or quantified in arbitrary units with respect to, or measured as multiples of, a predefined standard or reference point/pattern.

A method is provided for identifying a pattern of brain activity associated with a placebo effect response to a placebo treatment for a disease or disorder comprising: determining, by positron emission tomography or magnetic resonance imaging (MRI) in a subject receiving, or who has received, the placebo treatment for the disease or disorder, functional activity at each of a plurality of coordinates of the subject's brain during at least two different time points and identifying, through spatial co-variance analysis of the functional activity, which coordinates show a consistent trend over the at least two different time points in functional activity correlating with a placebo effect response to the placebo treatment, so as to thereby determine a pattern of brain activity associated with a placebo effect response to a placebo treatment for a disease or disorder.

In an embodiment, the MRI is functional MRI (fMRI).

In an embodiment, the disease or disorder is a neurological disease or disorder. In an embodiment, the disease or disorder is a psychological disease or disorder.

In an embodiment, the pattern of brain activity associated with a placebo effect is not found in a subject who is receiving or who has received the placebo treatment but who does not exhibit an improvement in the disease or disorder.

In an embodiment, the pattern of brain activity associated with a placebo effect is not found in a subject who is receiving a test treatment for the disease or disorder but who does not exhibit an improvement in the disease or disorder, or is not found in a subject who has received a test treatment for the disease or disorder but who does not exhibit an improvement in the disease or disorder.

In an embodiment, the pattern of brain activity associated with a placebo effect is not found in a subject who is receiving a test treatment for the disease or disorder that is efficacious, or is not found in a subject who has received a test treatment for the disease or disorder that is efficacious.

In an embodiment, an improvement in the disease or disorder is determined by the subject exhibiting an improvement in at least one symptom of the disease or disorder or an improvement in at least one measurable parameter associated with the disease or disorder.

In an embodiment, the efficacious treatment for the disease or disorder improves at least one symptom of the disease or disorder or one measurable physical parameter associated with the disease or disorder

In an embodiment, the functional activities are, or have been, determined as showing a consistent trend over at least three different time points. In an embodiment, the consistent trend is a monotonic ordinal trend. In an embodiment, the spatial co-variance analysis is linearly-independent spatial co-variance analysis. In an embodiment, the coordinates are three-dimensional coordinates.

In an embodiment, the disease or disorder is a neurodegenerative disease. In an embodiment, the disease is Parkinson's disease. In an embodiment, improvement in at least one symptom of the disease or improvement in at least one measurable parameter associated with the disease or disorder is assessed by a Unified Parkinson's Disease Rating Scale (UPDRS).

In an embodiment, the disease or disorder is a neurodevelopmental disease. In an embodiment, the disease or disorder is a psychological disorder.

In an embodiment, the methods further comprise determining the efficacy of a test treatment for the disease or disorder on one or more subjects by assessing if an improvement occurs in one or more symptoms of, or measurable parameter of, the disease or disorder the disease or disorder during or subsequent to administration of the test treatment to the subject, wherein a test treatment associated with an improvement in a subject not exhibiting the pattern of brain activity associated with a placebo effect is an efficacious treatment in that subject.

In an embodiment, a test treatment associated with an improvement in one or more symptoms of, or measurable parameter of, the disease or disorder during or subsequent to administration of the test treatment to the subject, wherein the subject exhibits the pattern of brain activity associated with a placebo effect, is not considered in an efficacious treatment in that subject. The test treatment is the treatment being investigated for its efficacy, as opposed to the sham or placebo treatment.

In an embodiment of the methods, the subject is not receiving any other treatment known to be efficacious in treating the disease or disorder. For example, in an embodiment the subject is not receiving any anti-parkinsonian medications.

Placebo treatments are well known in the art and are used to mirror a test treatment, for which the placebo treatment is a sham treatment control. As used herein, a placebo treatment is such an intervention, such as administration of a composition not comprising the test agent or an active agent, or such as a surgical procedure which otherwise mirrors the a test surgical procedure to, for example, implant an active agent, but without implanting the active agent. One of skill in the art understands suitable placebos for a given intervention, and such are routinely determined and used in the art, for example in clinical trials.

As used herein, “improves” or “improvement in”, with regard to a disease, disorder or symptom thereof, or measurable parameter thereof, means a change in the disease, disorder or symptom thereof, or measurable parameter thereof, towards the non-disease state or non-disorder state, as applicable.

A parameter may be any parameter which is known to change in the disease or disorder, as compared to the non-disease or non-disorder state, respectively. Such parameters may be measured by techniques known in the art, such as, in non-limiting examples, by assessing movement initiation, shake, movement cessation, cognition parameter measurement, memory, physical indicators such as protein levels in CSF, blood, blood pressure. Symptom or disease improvement may also be assessed by using known techniques, for example Unified Parkinson's Disease Rating Scale (UPDRS) for Parkinson's disease or MDS-UPDRS, a depression rating scale for depression, such as Hamilton Depression Rating Scale or Raskin Depression Rating Scale.

A method is provided for determining efficacy of a candidate treatment, administered to a subject having a neurological or psychological disease, on a rate of progression of the disease comprising:

a) determining, by positron emission tomography or functional magnetic resonance imaging (fMRI) during administration of or after administration of the candidate treatment to the subject, functional activity at each of a plurality of predetermined coordinates of the subject's brain so as to determine a first pattern of activity, which coordinates have previously been identified through spatial co-variance analysis of functional activity as determined by positron emission tomography or fMRI in the brain of the subject or in the brain(s) of one or more other subjects suffering from the neurological or psychological disease during at least two different time points while the subject was, or subjects were, exhibiting the symptom or disease as showing a consistent trend in functional activity which correlates with worsening of the disease; and
b) comparing the first pattern of activity determined in step a) with a previously determined baseline pattern of activity,
wherein an expression of the first pattern of activity lower than the previously determined baseline pattern of activity indicates that the candidate treatment is efficacious in reducing the rate of progression of the disease, and wherein an expression of the first pattern of activity higher than the previously determined baseline pattern of activity indicates that the candidate treatment is not efficacious in reducing the rate of progression of the disease.

In an embodiment, the baseline pattern of activity is determined through identifying a plurality of coordinates through spatial co-variance analysis of functional activity, as quantified by positron emission tomography or fMRI in the brain of the subject or in the brain(s) of one or more other subjects suffering from the neurological or psychological disease during at least two different time points while the subject was, or subjects were, exhibiting the symptom or disease, which coordinates show a consistent trend in functional activity which correlates with worsening of the disease. In an embodiment, the method is used to determine the efficacy of the candidate treatment in a clinical trial.

In an embodiment, the method further comprises determining the baseline pattern of activity.

In an embodiment the method further comprises, prior to step a) identifying through spatial co-variance analysis of functional activity as determined by positron emission tomography or fMRI in the brain of the subject or in the brain(s) of one or more other subjects suffering from the neurological or psychological disease during at least three different time points while the subject was, or subjects were, exhibiting the symptom or disease, as showing a consistent trend in functional activity which correlates with worsening of the symptom or disease.

In an embodiment, the linearly independent spatial co-variance analysis is a supervised principal component analysis.

In an embodiment, the linearly independent spatial co-variance analysis is an ordinal trends canonical variates analysis.

A method is also provided for identifying a pattern of brain activity specifically associated with a symptom of a neurological or psychological disease comprising: determining, by positron emission tomography or functional magnetic resonance imaging (fMRI) in a subject exhibiting the symptom, functional activity at each of a plurality of coordinates of the subject's brain during at least two different time points and identifying, through spatial co-variance analysis of the functional activity, which coordinates show a consistent trend over the at least two different time points in functional activity correlating with the symptom, so as to determine a baseline pattern of activity specifically associated with the symptom of the neurological or psychological disease.

A method is also provided for identifying a pattern of brain activity specifically associated with worsening of a symptom of a neurological or psychological disease comprising:

determining, by positron emission tomography or functional magnetic resonance imaging (fMRI) in a subject exhibiting the symptom, functional activity at each of a plurality of coordinates of the subject's brain during at least two different time points and identifying, through spatial co-variance analysis of the functional activity, which coordinates show a consistent trend over the at least two different time points in functional activity correlating with the worsening of the symptom,
so as to thereby determine a baseline pattern of activity specifically associated with the worsening of symptom of the neurological or psychological disease.

A method is also provided for identifying a pattern of brain activity specifically associated with efficacious treatment of a symptom of a neurological or psychological disease comprising:

determining, by positron emission tomography or functional magnetic resonance imaging (fMRI) in a subject exhibiting the symptom and being treated with a treatment efficacious for that symptom, functional activity at each of a plurality of coordinates of the subject's brain during at least two different time points and identifying, through spatial co-variance analysis of the functional activity, which coordinates show a consistent trend over the at least two different time points in functional activity correlating with efficacious treatment of the symptom, so as to thereby determine a baseline pattern of activity specifically associated with efficacious treatment of the symptom of the neurological or psychological disease.

A method is also provided for identifying a pattern of brain activity specifically associated with a pre-phenoconversion state of a neurological disease comprising:

determining, by positron emission tomography or functional magnetic resonance imaging (fMRI) in a subject in a pre-phenoconversion state, functional activity at each of a plurality of coordinates of the subject's brain during at least two different time points and identifying, through spatial co-variance analysis of the functional activity, which coordinates show a consistent trend in functional activity over the at least two different time points correlating with the pre-phenoconversion state, so as to thereby determine a pattern of brain activity specifically associated with the pre-phenoconversion state of the neurological disease.

In an embodiment, the pattern of brain activity is not found in a subject who is not in a pre-phenoconversion state of the neurological disease.

A method is also provided for identifying a pattern of brain activity specifically associated with predisposition to a neurological disease comprising:

determining, by positron emission tomography or functional magnetic resonance imaging (fMRI) in a subject predisposed to the neurological disease, functional activity at each of a plurality of coordinates of the subject's brain during at least two different time points and identifying, through spatial co-variance analysis of the functional activity, which coordinates show a consistent trend in functional activity over the at least two different time points correlating with predisposition to the neurological disease,
so as to thereby determine a pattern of brain activity specifically associated with predisposition to a neurological disease.

In an embodiment, the pattern of brain activity is not found in a subject who is not predisposed to the neurological disease.

A method is also provided of determining a pre-phenoconversion subject as likely to phenoconvert to a neurological disease within a predetermined time period comprising determining, by positron emission tomography or functional magnetic resonance imaging (fMRI), functional activity at each of a plurality of predetermined coordinates of the pre-phenoconversion subject's brain so as to determine a first pattern of activity, and comparing the first pattern of activity to a baseline pattern of activity which correlates with a pre-phenoconversion state and does not correlate with a phenoconversion state,

wherein an expression of the first pattern of activity in excess of a predetermined multiple of the baseline pattern of activity indicates that the subject is likely to phenoconvert to the neurological disease within the predetermined time period, and wherein an expression of the first pattern of activity lower than a predetermined multiple of the baseline pattern of activity indicates that the subject is not likely to phenoconvert to the neurological disease within the predetermined time period.

In an embodiment, the predetermined time period is 1-25 years. In an embodiment, the predetermined time period is 1-5 years, 1-10 years, 1-15 years, or 1-20 years. In an embodiment, the predetermined time period is 5-10 years, 5-15 years, 5-20 years, or 5-25 years. In an embodiment, the predetermined time period is 10-15 years, 10-20 years, or 10-25 years. In an embodiment, the predetermined time period is 15-20 years, 15-25 years or 20-25 years.

In an embodiment of the methods, the neurological disease is Huntington's disease. In an embodiment, the subject has an autosomal dominant mutation on either of the subject's two copies of the Huntingtin gene.

A method is also provided for identifying a pattern of brain activity specifically associated with a placebo effect response to a placebo treatment for a disease or disorder comprising:

determining, by positron emission tomography or functional magnetic resonance imaging (fMRI) in a subject receiving or who has received the placebo treatment for the disease or disorder functional activity at each of a plurality of coordinates of the subject's brain during at least two different time points and identifying, through spatial co-variance analysis of the functional activity, which coordinates show a consistent trend over the at least two different time points in functional activity correlating with a placebo effect response to the placebo treatment, so as to thereby determine a pattern of brain activity specifically associated with a placebo effect response to a placebo treatment for a disease or disorder.

In an embodiment of the methods, the disease or disorder is a neurological disease or disorder.

In an embodiment, the pattern of brain activity is not found in a subject who is receiving or who has received an efficacious treatment for the disease or disorder.

In an embodiment of the methods, the functional activities are, or have been, determined as showing a consistent trend over at least three different time points.

In an embodiment of the methods, the consistent trend is a monotonic ordinal trend.

In an embodiment of the methods, the method is for determining efficacy of a candidate treatment on the rate of progression of a neurological disease.

In an embodiment of the methods, the spatial co-variance analysis is linearly-independent spatial co-variance analysis.

In an embodiment of the methods, the coordinates are three-dimensional coordinates.

In an embodiment of the methods, the neurological disease is a neurodegenerative disease.

In an embodiment of the methods, the neurological disease is a neurodevelopmental disease.

In an embodiment of the methods, each set of predetermined coordinates has a single numerical value corresponding to functional activity.

In an embodiment of the methods, each set of coordinates corresponds to a volume of interest in a subject's brain.

In an embodiment of the methods, each volume of interest is no greater than 1 cm³.

In an embodiment of the methods, the subject is predetermined to be suffering from a neurological disease, be in a prephenoconversion state of a neurological disease or be predisposed to a neurological disease.

In an embodiment the methods further comprise identifying the subject as suffering from a neurological disease, being in a prephenoconversion state of a neurological disease or being predisposed to a neurological disease.

In an embodiment of the methods, the subject has, or the subjects have, Parkinson's disease.

In an embodiment of the methods, the subject has, or the subjects have, Huntington's disease.

In an embodiment of the methods, the subject has, or the subjects have, Alzheimer's disease.

In an embodiment of the methods, the subject has, or the subjects have, obsessive-compulsive disorder or Tourette's syndrome.

In an embodiment of the methods, the subject is, or the subjects are clinically depressed.

In an embodiment of the methods, the coordinates have previously been identified through spatial co-variance analysis of a plurality of functional activities as determined by positron emission tomography in the brain of the subject.

In an embodiment of the methods, the coordinates have previously been identified through spatial co-variance analysis of a plurality of functional activities as determined by fMRI in the brain of the subject.

In an embodiment of the methods, the first pattern or activity is determined from activities showing a consistent trend over at least three different time points.

In an embodiment of the methods, the subject is a mammal. In an embodiment of the methods, the subject is a mammal a non-human primate. In an embodiment of the methods, the mammal is a human.

In an embodiment of the methods, one or more steps of the method is performed using one or more processors, and/or accessing one or more sets of data from a database using the one or one or more processors.

A system is provided for identifying related proteins, comprising: one or more data processing apparatus; and a computer-readable medium coupled to the one or more data processing apparatus having instructions stored thereon which, when executed by the one or more data processing apparatus, cause the one or more data processing apparatus to perform one of any of the above-described methods.

A computer-readable medium is provided comprising instructions stored thereon which, when executed by a data processing apparatus, causes the data processing apparatus to perform a method of one of any of the above-described methods.

Embodiments of the invention and all of the functional operations described in this specification can be implemented in digital electronic circuitry, or in computer software, firmware, or hardware, including the structures disclosed in this specification and their structural equivalents, or in combinations of one or more of them. Embodiments of the invention can be implemented as one or more computer program products, i.e., one or more modules of computer program instructions encoded on a computer readable medium for execution by, or to control the operation of, data processing apparatus. The computer readable medium can be a machine readable storage device, a machine readable storage substrate, a memory device, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The methods, or portions thereof, processes and logic flows described in this specification can be performed by one or more programmable processors executing one or more computer programs to perform functions by operating on input data and generating output. The methods, or portions thereof, processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device. Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the invention can be implemented on a computer having a display device, e.g., in non-limiting examples, a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Embodiments of the invention can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the invention, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other.

The methods as described herein can be applied wherein the consistent trend is a positive consistent trend, or, mutatis mutandis, wherein the consistent trend is a negative consistent trend.

In embodiments, the methods as described herein can each be applied as stated except for the substitution of an alternative brain activity imaging/quantification method in place of the recited PET and fMRI methods, for example, SPECT, CT. In embodiments the methods further comprise administering to the subject one or more agents, e.g. radionuclides, necessary to perform the brain activity imaging/quantification. In an embodiment, any two or more of the brain activity imaging/quantification methods can be used together to provide the detail on which the pattern of brain activity is identified. PET images demonstrate the metabolic activity chemistry of brain. A radiopharmaceutical, such as fluorodeoxyglucose, which includes both sugar and a radionuclide, is injected into the subject, and its emissions are measured by a PET scanner. The PET system detects pairs of gamma rays emitted indirectly by the positron-emitting radionuclide (tracer), which is introduced into the body on a biologically active molecule. Radiopharmaceuticals such as fluorodeoxyglucose as the concentrations imaged can be used as indication of the metabolic activity at that point. Magnetic resonance imaging (MRI) makes use of the property of nuclear magnetic resonance (NMR) to image nuclei of atoms inside the body, in this instance the brain. Strong magnetic field gradients cause nuclei at different locations to rotate at different speeds. 3-D spatial information can be obtained by providing gradients in each direction. In the embodiment of functional MRI (fMRI), the scan is used to measure the hemodynamic response related to neural activity in the brain.

A method is also provided for determining efficacy of a candidate treatment, administered to a subject having a neurological disease, on a rate of progression of a neurological disease comprising:

a) determining, by positron emission tomography or functional magnetic resonance imaging (fMRI) during administration of or after administration of the candidate treatment to the subject, functional activity during at least two different time points at each of a plurality of predetermined coordinates of the subject's brain (i.e., a “pattern”) which coordinates have previously been identified through spatial covariance analysis of functional activity as determined by positron emission tomography or fMRI in the brain of subjects suffering from the neurological disease during at least two time points correlating with appearance or worsening of disease; and
b) comparing changes in the functional activity determined during administration of or after administration of the candidate treatment wherein a reduction in the expression of the pattern during administration of or after administration of the candidate treatment compared to the previously determined baseline pattern expression value indicates that the candidate treatment is efficacious in treating the neurological disease.

A method is also provided for determining a pattern of brain activity associated with a placebo treatment in a subject or subjects comprising:

a) identifying, by spatial covariance analysis, a plurality of functional activities exhibiting a consistent trend over the at least two time points which correlate with the placebo treatment, thereby identifying a pattern of brain activity associated with the placebo treatment.
b) determining, by positron emission tomography or fMRI, functional activity at each of a plurality of predetermined coordinates of the brain in a plurality of subjects, during at least two different time points, while or before which the subjects are, exposed to the placebo treatment. In an embodiment, the method further comprises administering a candidate treatment to a subject and determining, by positron emission tomography or fMRI, during administration of or after administration of the treatment to the subject, functional activity during at least two time points at each of the plurality of predetermined coordinates of the brain showing the change in the pattern determined as associated with the placebo treatment, and comparing the expression of the pattern in that subject with the changes determined to be associated with the placebo treatment,
wherein replication of the trend associated with the placebo treatment during administration of or after administration of the treatment indicates that the candidate treatment is not different from placebo treatment (not efficacious), and wherein no replication of the trend associated with the placebo treatment during administration or after administration of the treatment does not indicate that the candidate treatment is not efficacious.

A method is also provided for determining the expression of a pattern of brain activity in a subject having a genetic mutation rendering the subject susceptible to developing a neurological disease which pattern of brain activity is associated with a pre-phenoconversion state of the neurological disease comprising:

a) identifying, by spatial covariance analysis, a plurality of functional activities exhibiting a consistent trend over at least two time points and which correlate with the pre-phenoconversion state of the disease, thereby identifying the pattern of brain activity associated with the pre-phenoconversion state of the neurological disease.
b) determining, by positron emission tomography or fMRI, functional activity at each of a plurality of predetermined coordinates of the brain of subjects having the genetic mutation during at least two different time points during which the subject is, [and to what degree] in a pre-phenoconversion state of the neurological disease; and

A method is also provided for determining a pattern of brain activity associated with a symptom of a multi-symptom disease comprising:

a) identifying, by spatial covariance analysis, a pattern in a plurality of functional activity exhibiting a consistent trend over the at least two time points and which correlate with the presence and/or severity of the symptom, thereby identifying the pattern of brain activity associated with a particular symptom of the multi-symptom disease.
b) determining, by positron emission tomography or fMRI, functional activity at each of a plurality of predetermined coordinates of the brain pattern during at least two time points while or before which the subjects are, exhibiting one of the symptoms of the multi-symptom disease. In an embodiment, the method further comprises administering a candidate treatment for the symptom to a subject and determining, by positron emission tomography or fMRI, during administration of or after administration of the candidate treatment for the symptom to the subject, functional activity during at least two time points at each of the plurality of predetermined coordinates of the brain pattern showing the consistent trend in functional activity determined as associated with the symptom, and comparing the functional activity so determined with that associated with the symptom,
wherein reversal of, or reduction of, pattern expression during or after administration of the candidate treatment as compared to that associated with the baseline presence of the symptom indicates that the candidate treatment is efficacious in treating that particular symptom and wherein an increase or no change in the expression of pattern during or after administration of the candidate treatment as compared to the baseline expression of the pattern indicates that the candidate treatment is not efficacious in treating the symptom of the multi-symptom disease.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### Experimental Results I

### Introduction

Resting tremor is one of the cardinal features of Parkinson's disease (PD) and is present in 75 to 100% of patients during the course of the illness (Rajput et al., 1991; Hughes et al., 1993). The pathophysiology of parkinsonian tremor is thought to be distinct from that of akinesia and rigidity, the other major clinical symptoms of the disease (e.g., Fishman, 2008; Zaidel et al., 2009). For instance, in PD, loss of nigral dopaminergic projections to the putamen correlates consistently with clinical ratings of akinesia and rigidity but not tremor (Eidelberg et al., 1995a; Benamer et al., 2003). Moreover, unlike akinetic-rigid manifestations of the disease, parkinsonian tremor is not uniformly responsive to dopaminergic therapy. Indeed, nigrostriatal dopaminergic loss appears to be a necessary but insufficient condition for the development of PD tremor (Fishman, 2008; Zaidel et al., 2009).

The ventral intermediate (Vim) nucleus of the thalamus has traditionally been regarded as the optimal target for the surgical relief of tremor (e.g., Machado et al., 2006). Neurons in this region receive projections from the deep cerebellar nuclei and discharge in synchrony with parkinsonian tremor (Lenz et al., 1994). Given that PD tremor can also be alleviated by lesions of other brain regions, including the pons and cerebellum (Boecker and Brooks, 1998), the Vim thalamic nucleus can be viewed as one of several interconnected nodes of a spatially distributed tremor circuit. Nevertheless, the precise anatomical/functional topography of this large-scale network is not known, particularly with respect to the relative contributions of the basal ganglia and cerebellum to this pathway (e.g., Volkmann et al., 1996; Deuschl et al., 2001; Timmermann et al., 2003; 2007; Zaidel et al., 2009). The functional imaging hallmarks of parkinsonian tremor are also not fully defined, particularly from the circuit standpoint. Resting state imaging of glucose metabolism with ¹⁸F-fluorodeoxyglucose (FDG) positron emission tomography (PET) has provided a useful means of assessing disease-related changes in brain function at the network level (Eidelberg, 2009). Patient expression of a previously validated PD-related metabolic covariance pattern (PDRP) (Ma et al., 2007; Eidelberg, 2009) has been found to correlate with clinical ratings for akinesia and rigidity but not tremor (Eidelberg et al., 1994; 1995b; Feigin et al., 2001; Lozza et al., 2004). Moreover, PDRP expression has been found to be elevated to similar levels in PD patients with comparable degrees of bradykinesia, whether or not tremor is also present (Isaias et al., 2010; cf. Antonini et al., 1998). That said, the characterization of a specific metabolic network associated with PD tremor has been particularly challenging because of the much smaller signal associated with this disease manifestation. An earlier study (Antonini et al., 1998) sought to identify a significant PD tremor network that was independent of the dominant PDRP metabolic abnormalities. The analytical strategy that was used was cross-sectional, in that the tremor-related pattern was sought in FDG PET data from a combined group of patients with tremor and akinetic-rigid dominant symptoms. However, consistent with the relatively small effect of tremor on composite ratings of motor disability in PD (Martinez-Martin et al., 1994; Stochl et al., 2008), the signal associated with the corresponding metabolic network proved insufficient for prospective application.

Herein, the problem using a novel within-subject strategy in which tremor dominant PD patients underwent FDG PET scanning at baseline and again during deep brain stimulation (DBS) of the ventral intermediate (Vim) thalamic nucleus. Using a new voxel-based network approach (Habeck et al., 2005; Habeck and Stern, 2007; Carbon et al., 2010), a distinct PD tremor-related metabolic pattern (PDTP) was identified that was sufficiently stable to be applied on a prospective single case basis. The validity of PDTP expression as a quantitative network-based descriptor of this disease manifestation was demonstrated by the excellent reproducibility of this objective network measure, its consistent correlation with independent clinical tremor ratings, and its significant progression over time. Also assessed was the use of the PDTP for modulation by interventions directed specifically at this symptom.

### Materials and Methods

Pattern identification: Nine PD patients were studied (8 men and 1 woman, age 65.9±9.6 years [mean±SD], off-state Unified Parkinson's Disease Rating Scale (UPDRS) motor ratings 36.6±14.2) who underwent clinically effective Vim DBS for tremor dominant symptoms (Table 3). Motor manifestations of PD were considered to be tremor dominant if the summed limb UPDRS tremor scores were ≧4 (items 20 and 21), with at least one limb scoring ≧2 (Antonini et al., 1998; Isaias et al., 2010). In this group, the stimulation parameters were: voltage 3.0±0.6 (V); pulse width 100±42.4 (μs); stimulation frequency 160±24.2 (Hz). Seven of the nine patients exhibited predominant tremor on the right side and had a stimulator placed unilaterally in the left Vim thalamic nucleus; the remaining two patients exhibited tremor dominant symptom on both the right and left body sides and underwent bilateral electrode implantation. Cerebral blood flow (H2 15O PET) data from these subjects have appeared previously (Fukuda et al., 2004).

Metabolic imaging: The patients were scanned on two consecutive days in random order. On the first day, the stimulators were switched off (OFF) approximately 3 hours prior to PET; the stimulators were switched on after scanning. On the next day, scanning was conducted with the stimulator on (ON), with settings determined by the maximal tremor suppression that was achieved without pain or adventitious movements. Before each PET session, the patients fasted overnight; parkinsonian medications were withheld for at least 12 hours before imaging. In each PET session, the subjects were rated according to the UPDRS (Fahn S and Elton R, 1987) approximately 1 hour before imaging. In addition to a composite motor rating (the sum of items 18-31), separate subscale ratings for tremor (the sum of items 20 and 21) and akinesia/rigidity (the sum of items 18, 19, 22, and 27-31) were obtained. Moreover, in seven of the patients, triaxial accelerometry (TRIAX) was used to measure tremor amplitude and frequency in the upper limbs contralateral to Vim stimulation. The details of the TRIAX recording procedures and data analysis are provided elsewhere (Fukuda et al., 2004). In each PET session (i.e., on and off stimulation), TRIAX recordings were acquired for at least 10 minutes to assure physiological stability (<5% variability) of the measured parameters during imaging.

FDG PET was performed in three dimensional (3D) mode using the GE Advance tomograph (General Electric Medical Systems, Milwaukee, Wis.) at North Shore University Hospital; the details of these procedures have been provided elsewhere (Ma et al., 2007). The studies were performed with the subjects' eyes open in a dimly lit room and with minimal auditory stimulation. Ethical permission for the PET studies was obtained from the Institutional Review Board of North Shore University Hospital. Written consent was obtained from each subject after detailed explanation of the procedures. Scan preprocessing was performed as described elsewhere (Huang et al., 2007b). In the two bilateral Vim DBS patients, images from the right hemisphere were flipped so that the operated side appeared on the left, along with the other stimulated hemispheres. Individual images were nonlinearly warped into Talairach space using a standard PET template, and smoothed with an isotropic Gaussian kernel (10 mm) in all directions to improve the signal-to-noise ratio.

Pattern derivation: To identify a specific metabolic brain network associated with PD tremor, the on and off stimulation FDG PET scans were analyzed from the nine Vim DBS patients using Ordinal Trends Canonical Variates Analysis (OrT/CVA) (Habeck et al., 2005; Moeller and Habeck, 2006) (software available at groups.google.com/group/gcva). OrT/CVA is a form of supervised principal component analysis (PCA) (Bair E, 2006) designed to identify linearly independent spatial covariance patterns for which subject expression increases (or decreases) in as many individuals as possible across scan conditions. OrT/CVA differs from voxel-wise univariate contrasts in that it requires that pattern expression exhibit an “ordinal trend”, the property of consistent change across conditions on a subject-by-subject (rather than on a group mean) basis. In addition to the identification of relevant spatial covariance patterns in the data, OrT/CVA quantifies the expression of the pattern(s) in each subject and condition. The significance of candidate patterns is assessed by permutation tests of the pattern expression measures (i.e., the principal component (PC) scalars or subject scores) to exclude the possibility that the observed changes across subjects/conditions had occurred by chance. Likewise, the reliability of the regional contributions to the candidate pattern (i.e., the voxel weights) is assessed using bootstrap estimation procedures (Habeck and Stern, 2007).

In the current study, a significant PD tremor-related metabolic pattern (PDTP) was sought among the linearly independent spatial covariance patterns (i.e., the orthogonal PCs) resulting from OrT/CVA of the scans acquired on and off Vim stimulation. The following model selection criteria were applied to the individual patterns: (1) the analysis was limited to the first 6 PCs, which typically account for at least 75% of the subject×region variance (Habeck and Stern, 2007); (2) subject scores for these PCs were entered singly and in all possible combinations into a series of logistic regression models, with stimulation condition (OFF, ON) as the dependent variable and the subject scores for each set of PCs as the independent variables for each model. The best model was considered to be that with the smallest Akaike information criterion (AIC) value. The selected PC(s) in this model were then used in linear combination to yield the spatial covariance pattern that was most closely related to the difference across stimulation conditions. The resulting pattern was considered to exhibit a significant ordinal trend if the associated subject scores differed from chance at p<0.05 (permutation test). To establish that the candidate pattern was indeed tremor-related subject scores measured in the baseline off-stimulation condition (i.e., without tremor suppression) were correlated with the simultaneously recorded TRIAX measurements. These correlations were assessed using regression analysis, with and without including DBS voltage as a covariate.

OrT/CVA covariance map(s) were displayed at a voxel weight threshold of Z=2.70, p<0.01 with a cluster cutoff of 50 voxels. Regions contributing to the pattern were considered significant for p<0.05 on bootstrap estimation. Because the tremor-related pattern was identified in the analysis of hemispheric PET data from predominantly unilateral Vim stimulation cases, the associated voxel weights were flipped to produce a symmetrical brain network for the quantification of pattern expression in whole-brain scan data from prospective subjects. The degree of similarity/difference between the PDTP and PDRP metabolic topographies was also determined by computing the variance shared (r2) between all the corresponding non-zero voxel weights on the two pattern images. Likewise, the PDTP topography was compared to that of a recently described normal movement-related covariance pattern (NMRP), identified using OrT/CVA of motor activation responses from healthy subjects (Carbon et al., 2010). In these analyses, the two pattern images (i.e., PDTP and PDRP; PDTP and NMRP) were spatially normalized and only voxels that differed from zero in both images were considered. Voxels from each pattern image were formatted into a single vector by appending successive rows in each plane of the image. The two vectors were then entered input into the MATLAB statistical routine ‘corr’ to calculate the correlation coefficient (r).

Pattern validation: Next a series of single case computations was performed to quantify PDTP and PDRP expression in prospective imaging datasets. The resulting network values (subject scores) were correlated with UPDRS subscale ratings for tremor and akinesia/rigidity. All PDTP and PDRP scores were Z-transformed with respect to values from 20 age-matched healthy control subjects (11 men and 9 women, age 60.6±13.0 years) so the control group for each network had a mean value of zero and a standard deviation of one. These forward analyses were performed using an automated voxel-wise procedure (available at www.fillon.ucl.ac.uk/spm/ext/#SSM) as described in detail elsewhere (Ma et al., 2007; Spetsieris et al., 2009).

1. The test-retest reliability of prospectively computed PDTP subject scores was determined. PDTP expression was quantified in 14 PD patients (7 men and 7 women; age 64.1±8.9 years; motor UPDRS 22.0±14.5; Table 1) who underwent repeat FDG PET imaging (Asanuma et al., 2006). Within-subject reproducibility of PDTP values in this group was assessed by computing the intraclass correlation coefficient (ICC) (Ma et al., 2007).

2. To determine the specificity of PDTP scores for parkinsonian tremor, the expression of this pattern was quantified in 41 subsequent PD patients (31 men and 10 women, age 59.8±9.1 years, motor UPDRS 27.7±16.2; Table 1) who underwent FDG PET in the off-medication state. Computed PDTP scores for these subjects were correlated with UPDRS subscale ratings for tremor and akinetic-rigidity using multiple linear regression; disease duration and subject age and gender were used as covariates in this analysis. By including both subscale ratings and the PDTP scores in a single multiple regression model (West et al., 1996), the magnitude of PDTP correlations was directly contrasted with tremor vs. akinesia/rigidity.

3. Whether parkinsonian tremor was associated with elevated PDTP values measured was determined using functional imaging modalities other than FDG PET. PDTP expression was quantified in 18 other PD patients (14 men and 4 women, age 63.1±7.0 years, motor UPDRS 34.0±13.1; Table 1) who underwent technetium-99methylene cysteine dimmer single photon emission computed tomography (^{[99m]}Tc-ECD SPECT) perfusion imaging in the off-medication state (Isaias et al., 2010). Nine of these subjects were classified as tremor predominant; the others were classified as akinetic-rigid predominant with little or no tremor. Prospectively computed PDTP scores for these patients were compared to corresponding values from nine healthy control subjects (5 men and 4 women, age 73.2±5.6 years) who also underwent ECD SPECT. This analysis was conducted using one-way analysis of variance (ANOVA) with post-hoc Bonferroni tests. Because the healthy control subjects were older (p<0.05, Student's t-test) than the patients one-way analysis of covariance (ANCOVA) was employed to adjust for the age difference.

TABLE 1
Demography of PD groups for validation and healthy control group

| | Test- | | Prospective | | Prospective | | |
|---|---|---|---|---|---|---|---|
| | Retest | | (FDG | | (ECD | | Progression |
| | (FDG PET) | | PET) | | SPECT) | | (FDG PET) |
| n | 14 | | 41 | | 18 | | 15 |
| Age | 64.1 | (8.9)^{a} | 59.8 | (9.1) | 61.8 | (7.0) | 58.0 (10.2)^{c}/60.3 |
| | | | | | | | (10.0)^{d}/63.0(7.9)^{e} |
| M:F | 7:7 | | 31:10 | | 14:4 | | 11:4 |
| Disease | 3.8 | (5.3) | 9.4 | (6.3) | 8.5 | (2.9) | 2.1(0.6)^{d}/4.0(0.7)^{e} |
| duration | | | | | | | |
| UPDRS^{b} | 22.0 | (14.5) | 27.7 | (16.2) | 34.0 | (13.1) | 9.1(4.5)^{c}/14.8(4.3)^{d}/ |

18.6(5.0)^{e}

^{a}Mean ± SD
^{b}Composite UPDRS motor ratings in a baseline state obtained 12 hrs after the cessation of antiparkinsonian medications
^{c}baseline in progressive PD group
^{d}24 months in progressive PD group
^{e}48 months in progressive PD group

Time course of pattern expression: To determine whether longitudinal changes in PDTP expression are sensitive to symptom progression, PDTP and PDRP scores were computed in FDG PET scans from 15 early stage PD patients (11 males and 4 females; age: 58.0±10.2 years; baseline motor UPDRS 8.2±4.5; Table 1) who participated in our previously reported longitudinal imaging study (Huang et al., 2007b; Tang et al., 2010). In all subjects, PDTP and PDRP scores were separately quantified at each time point (0, 24, 48 months). Longitudinal changes in tremor and akinesia/rigidity subscale ratings and concurrent changes in PDTP/PDRP expression were evaluated using one-way repeated measure analysis of variance (RMANOVA) Annualized rates of progression over the three time points were estimated for each measure using an individual growth model (Singer and Willett, 2003)

Effects of treatment on pattern expression: To determine whether therapeutic interventions directed at parkinsonian tremor are associated with PDTP network modulation, changes were assessed in PDTP/PDRP expression during STN DBS and compared the results to the corresponding network changes observed during Vim stimulation. The STN DBS cohort was comprised of nine different tremor dominant PD patients (7 men and 2 women; age 59.5±12.9 years; motor UPDRS ratings 32.3±13.3) with bilaterally implanted electrodes (Table 3). In this group, the stimulation parameters were: voltage 3.1±0.6 (V), pulse width 78±19.0 (αs), stimulation frequency 165±29.6 (Hz). As in the Vim DBS group, these patients underwent FDG PET in the ON and OFF conditions in separate consecutive day imaging sessions. In both the Vim and STN DBS groups, PDTP and PDRP scores were computed on an individual hemisphere basis in each stimulation condition (Tro\check{s}t et al., 2006; Tang et al., 2010). These calculations were performed using an automated voxel-wise algorithm (see above), blind to subject, DBS target (Vim, STN), and stimulation condition (OFF, ON).

Hemispheric changes in pattern expression with stimulation (ON−OFF) were compared with analogous changes (RETEST−TEST) measured in the 14 PD patients described above who underwent repeat FDG PET without intervention. In this control group, changes in pattern expression in each hemisphere were averaged and compared to the corresponding hemispheric changes measured in the two DBS treatment groups. Differences in network modulation (i.e., between-session changes in pattern expression) across the three groups (Vim DBS, STN DBS, control) were compared using one-way ANOVA followed by post-hoc Bonferroni tests. The network analyses were followed up with mass-univariate procedures to identify regions in which the two DBS interventions gave rise to similar metabolic changes (i.e., areas in which both Vim DBS and STN DBS led to either increases or decreases in regional glucose utilization). This was achieved using conjunction analysis in SPM5 (Friston et al., 2005); the results were considered significant at p<0.05 (family wise error [FWE]-corrected). All statistical analyses were performed using SPSS software (SPSS, Chicago, Ill.) and SAS 9.1 (SAS Institute Inc.), and were considered significant for p<0.05 (two-tailed).

### Results

Parkinson's Disease-Related Tremor Pattern

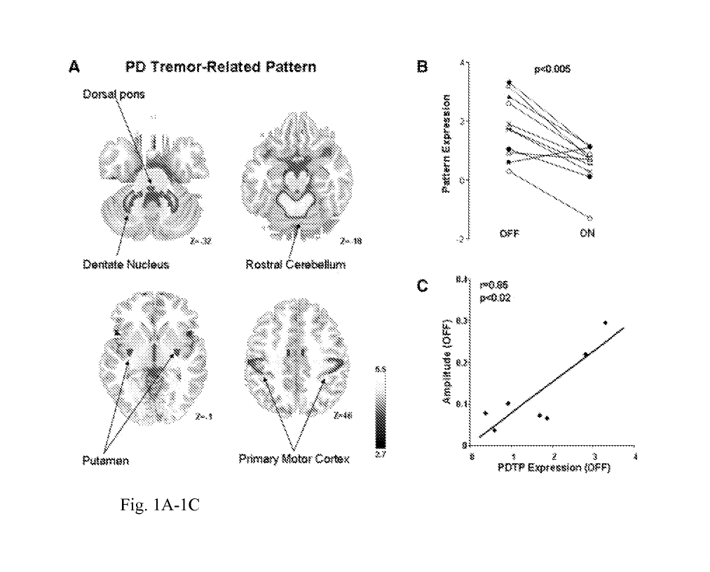

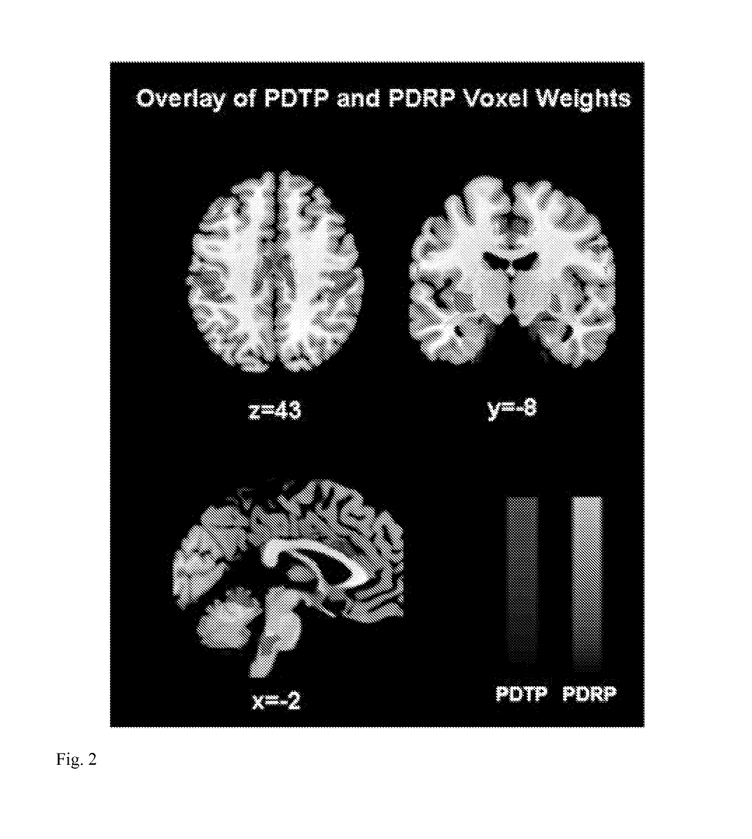

Network analysis of the FDG PET scans acquired on and off Vim stimulation revealed a significant spatial covariance pattern (FIG. 1A) characterized by increased metabolic activity in the anterior cerebellum (lobule IV-V) and dentate nucleus, primary motor cortex, and, to a lesser degree, in the caudate and putamen (Table 2). Voxel weights on the pattern were stable by bootstrap estimation (p<0.05). Voxel-wise correlation of the regional loadings on this pattern disclosed an 18% correspondence with the PDRP topography (FIG. 2) and no correspondence (0.01%) with the normal movement-related activation pattern (NMRP) topography.

TABLE 2
Regions contributing to Parkinson's disease tremor-related
metabolic pattern (PDTP)

| | Coordinates^{a} | | | |
|---|---|---|---|---|
| Regions | x | y | z | Zmax |
| Cerebellum (lobule TV/V)^{b} | 10 | −46 | −14 | 5.08*** |
| Dentate Nucleus | 14 | −40 | −32 | 3.25** |
| Putamen | −32 | −8 | 4 | 2.74* |
| Cingulate cortex (BA 24/32) | 0 | 24 | 24 | 3.71** |
| Sensorimotor cortex (BA 4/1, 2, 3) | −28 | −24 | 48 | 3.73** |

^{a}Montreal Neurological Institute (MNI) standard space.
^{b}According to the atlas of Schmahmann (Schmahmann et al., 2000).
*p < 0.01, **p < 0.001, ***p < 0.0001 (see text).

The expression of this pattern in the individual subjects (FIG. 1B) exhibited a significant ordinal trend (p<0.005, permutation test), in that network activity values declined with stimulation in 10/11 treated hemispheres. Moreover, in the baseline (OFF) condition, hemispheric pattern expression (FIG. 1C) correlated with concurrent TRIAX measurements of tremor amplitude in the contralateral upper limb (r=0.85, p<0.02). Nonetheless, tremor amplitude did not correlate with PDRP values measured in the same hemispheres (p=0.26). There was no correlation (p>0.26) between changes in pattern expression across conditions and individual differences in the stimulation parameters (DBS voltage and stimulation frequency) that were employed. Based upon the association of this spatial covariance pattern with parkinsonian tremor the bilateralized form of this metabolic network was termed the PD tremor-related pattern (PDTP).

Pattern Validation

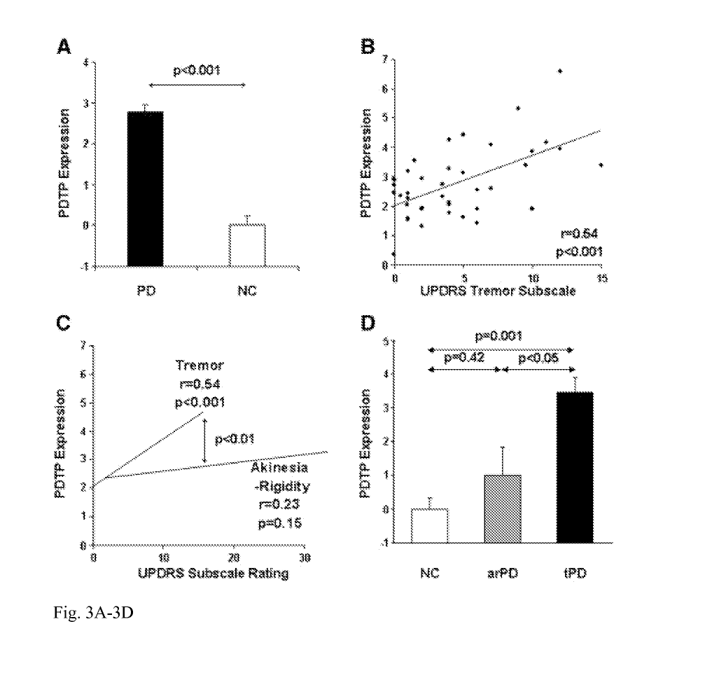

In an independent PD patient population (n=14), PDTP scores exhibited excellent test-retest reproducibility (ICC=0.86, p<0.0001) over an 8-week interval. PDTP expression was then quantified in another independent PD patient cohort (n=41) scanned with FDG PET, and the resulting network values were compared to those from the healthy volunteer subjects (n=20). It was found that the resulting PDTP scores (FIG. 3A) were abnormally elevated in this patient group (p<0.001, Student's t-tests). These values were found to correlate with UPDRS tremor subscale ratings (r=0.54, p<0.001; FIG. 3B). This correlation remained significant after adjusting for individual differences in disease duration, subject age and gender (r=0.56, p<0.001), as well as following the exclusion of the five subjects without clinically discernible tremor (r=0.56, p<0.001). Nonetheless, the correlation between PDTP expression and akinesia-rigidity subscale ratings was not significant (r=0.23, p=0.15) and was of smaller magnitude (p<0.01; multiple regression) than that observed with tremor ratings (FIG. 3C).

PDTP scores were also quantified in tremor and akinesia-rigidity dominant PD cohorts and in healthy volunteers scanned with ECD SPECT (n=9 in each group). A significant difference was found in pattern expression across the three groups (F_{(2,26)}=11.36, p<0.001; one-way ANOVA). Indeed, the tremor dominant patients exhibited increased PDTP expression (FIG. 3D) relative to their akinetic-rigid counterparts (p<0.02) as well as the healthy controls (p<0.001), while the PDTP expression did not differ (p=0.38) between the akinetic-rigid patients and healthy controls. The results remained significant following adjustment for group differences in age (whole model: p=0.001; tremor vs. akinetic-rigid: p<0.02; tremor vs. control: p=0.01; akinetic-rigid vs. control: p=0.99).

Effects of Disease Progression

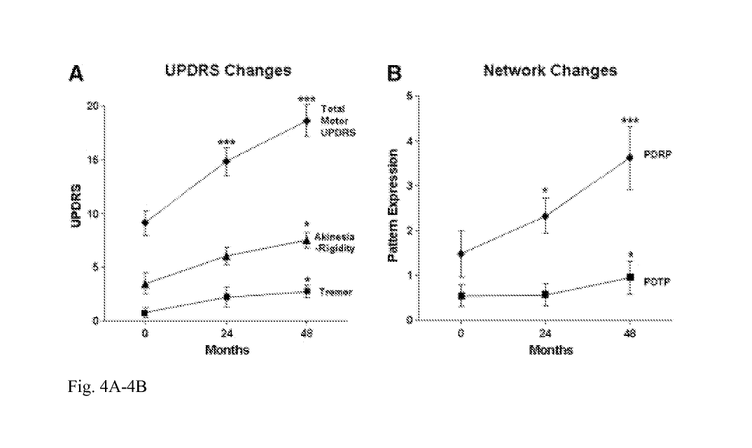

Longitudinal changes in UPDRS tremor and akinesia-rigidity subscale ratings were assessed (FIG. 4A), and the corresponding changes in PDTP and PDRP expression (FIG. 4B), in the disease progression cohort described above (see Methods). Over time, there was significant worsening in akinesia-rigidity (F_{(2, 12)}=5.6, p=0.02; one-way RMANOVA) and tremor (F_{(2, 15)}=6.4, p=0.01), corresponding to a progression rate of 0.95 points/year for the former (p<0.01, individual growth model) and 0.41 points/year for the latter (p<0.005). For both subscores, significant increases were present only at 48 months relative to baseline (p<0.05; post-hoc Bonferroni test). These changes paralleled with concurrent progression in the activity of both PD-related metabolic networks (PDTP: F_{(2, 23)}=4.67, p=0.01; PDRP: F_{(2,23)}=29.9, p<0.0001; one-way RMANOVA). The longitudinal time course was however different for the two patterns (interaction effect: F_{(2, 23)}=6.0, p<0.01; 2×3 RMANOVA), with PDTP expression progressing at a considerably slower rate (0.10 point/year, p<0.05; individual growth model) than the PDRP (0.51 point per year, p<0.0001). Relative to baseline, there were no changes in PDTP expression at 24 months (p=0.99; post-hoc Bonferroni test) and a significant increase at 48 months (p<0.05). By contrast, there were significant increases in PDRP expression at both the second (p<0.05) and third time points (p<0.0001) relative to baseline.

Effects of Treatment on Pattern Expression

The effects of stimulation on the total motor UPDRS and the tremor and akinesia-rigidity subscale ratings are summarized in Table 3. At baseline, total motor UPDRS ratings did not differ across the two DBS groups (p=0.56, Student's t-test). However, at baseline, tremor ratings were relatively greater for the Vim DBS group (p<0.05). Total motor UPDRS ratings and tremor subscale ratings declined with stimulation in both stimulation groups (p<0.01; paired Student's t-tests). By contrast, significant reduction in the akinesia-rigidity subscale ratings was evident only for the STN DBS group (p<0.05). Although reductions in the total motor UPDRS did not differ between interventions (p=0.62), the decline in tremor ratings was found to be greater for the Vim relative to the STN DBS groups (p<0.05).

TABLE 3
Clinical features of DBS patients

| | | | | | | Healthy |
|---|---|---|---|---|---|---|
| | | Vim DBS PD | | STN DBS PD | | Controls |
| n | | 9 | | 9 | | 20 |
| Age (years) | | 65.9 | (9.6)^{a} | 59.5 | (12.9) | 60.6 (13.0) |
| M:F | | 8:1 | | 7:2 | | 11:9 |
| Disease duration | | 8.6 | (4.5) | 9.7 | (4.2) | |
| UPDRS^{b} | | | | | | |
| Total motor | OFF | 36.6 | (14.2) | 32.3 | (13.3) | |
| | ON | 22.9 | (12.4) | 20.9 | (9.3) | |
| | Δ | −13.7 | (8.6)** | −11.4 | (9.2)** | |
| Tremor | OFF^{†} | 8.9 | (3.7) | 5.1 | (3.0) | |
| | ON | 1.6 | (2.9) | 1.9 | (1.3) | |
| | Δ^{†} | −7.3 | (4.1)** | −3.2 | (3.4)* | |
| Akinesia-Rigidity | OFF | 13.2 | (8.1) | 16.5 | (6.9) | |
| | ON | 10.6 | (5.4) | 10.6 | (5.5) | |
| | Δ | −2.6 | (3.7) | −5.9 | (7.6)* | |

^{a}Mean ± SD
^{b}Composite UPDRS motor ratings in the baseline off-stimulation (OFF) state and in the stimulated state (ON). Both treatment states were evaluated 12 hrs after the cessation of antiparkinsonian medications.
Δ: ON-OFF
Significant group differences (Student's t-test): ^{†}p < 0.05
Significant ON-OFF differences (paired t-test): *p < 0.05, **p < 0.01

Network Changes

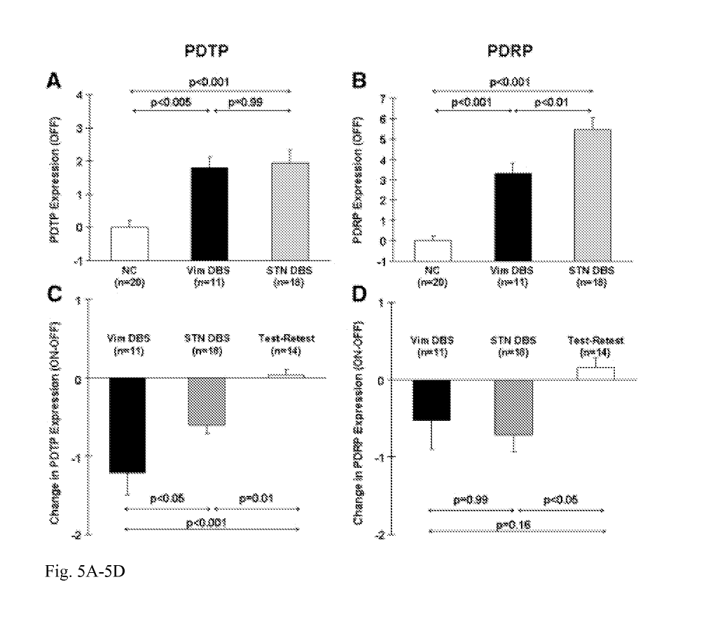

At baseline, there was evidence of a significant group difference in the expression of both PD-related metabolic patterns (PDTP: F_{(2,48)}=12.8, p<0.001; PDRP: F_{(2,48)}=45.4, p<0.001; one-way ANOVA). Baseline PDTP expression (FIG. 5A) was elevated relative to controls in both the Vim (p<0.002, post-hoc Bonferroni test) and the STN DBS cohorts (p<0.001). Baseline PDRP expression (FIG. 5B) was also abnormally elevated (p<0.001) in both patient groups, although these network values were relatively higher (p<0.008) in the STN DBS group. Significant differences in stimulation-mediated PDTP modulation (FIG. 5C) were observed across the three groups (F_{(2,42)}=13.6, p<0.001, one-way ANOVA), with greater changes in the stimulation groups relative to the test-retest controls (Vim DBS: p<0.001; STN DBS: p=0.01, post-hoc Bonferroni tests). The PDTP changes were greater in magnitude in the Vim DBS group relative to the STN DBS group (p=0.04). Significant group differences in stimulation-mediated PDRP modulation (FIG. 5D) were also noted (F_{(2,42)}=4.3, p=0.02). During STN stimulation, significant treatment-mediated changes in PDRP expression were evident with respect to test-retest controls (p=0.02, post-hoc Bonferroni test). Changes in PDRP expression were, however, not significant during Vim stimulation (p=0.16).

Regional Changes

Given that significant improvement in tremor ratings and PDTP suppression was observed with both Vim and STN stimulation, identity of the brain regions in which treatment mediated changes in metabolic activity occurred with both interventions was sought. Voxel-wise analysis of treatment-mediated metabolic changes in the two stimulation groups revealed a single, highly significant cluster in the sensorimotor cortex (SMC: x=40, y=−32, z=64; Zmax=6.01, p<0.05, FWE-corrected), corresponding to shared reductions (ON<OFF) in this region with both interventions. Post-hoc analysis revealed significant reductions in metabolic activity in this region during stimulation (Vim DBS: p<0.01; STN DBS: p<0.05, paired t-test). Metabolic activity in this cluster differed across the three groups (Vim DBS, STN DBS, healthy controls) in both the OFF and ON conditions (OFF: F_{(2,48)}=31.1, p<0.001; ON: F_{(2,48)}=15.0, p<0.001, one-way ANOVA).

Post-hoc analysis revealed that relative to the control group, regional metabolic activity at baseline was similarly elevated in both stimulation groups (p<0.001). During stimulation, metabolic activity in this region remained abnormally elevated in the STN DBS group. By contrast, during stimulation, the mean value for the Vim DBS group fell to within 1 SD of normal. No regions were identified in which treatment-mediated increases in metabolic activity were present with the two interventions.

### Discussion

In this study, an innovative covariance mapping approach was used to identify and validate a distinct tremor-related metabolic network in PD patients scanned on and off Vim thalamic stimulation. The PDTP was characterized by network-related increases in the metabolic activity of the cerebellum/dorsal pons and primary motor cortex, and to a lesser degree in the caudate/putamen. The expression of this pattern in individual patients correlated with independent clinical ratings for tremor, but not akinesia-rigidity. This contrasted with PDRP expression, which has been found to correlate with ratings for akinesia/rigidity, but not tremor (Eidelberg et al., 1994; 1995a; Antonini et al., 1998). Indeed, PDTP expression was selectively elevated in tremor dominant patients relative to their akinetic-rigid atremulous counterparts. Furthermore, the expression of this pattern increased with advancing disease, but at a slower rate than for the akinesia-related PDRP. Imaging studies of DBS interventions directed at parkinsonian tremor revealed significant reductions in PDTP expression during either Vim or STN stimulation. By contrast, significant PDRP modulation and concomitant improvement in akinesia/rigidity occurred only with STN stimulation. In aggregate, the findings suggest that the PDTP represents a distinct functional topography of PD, which may serve as a quantitative descriptor of the effects of antiparkinsonian interventions directed at tremor pathways. Moreover, the quantification of changes in PDTP and PDRP expression during treatment may help objectively parcellate the effects of novel antiparkinsonian therapies on the major motor manifestations of the illness.

The pathophysiology of parkinsonian tremor remains unclear. Convergent lines of evidence suggest that resting tremor in PD is not a direct reflection of dopamine deficiency (Fishman, 2008). Tremor has been found to be independent of other motor manifestations of the disease (see e.g., Eidelberg et al., 1994) and has a relatively small impact on the variability of clinical ratings data (Martinez-Martin et al., 1994; Stochl et al., 2008). This is consistent with the results of dopaminergic imaging studies. In contrast to akinesia and rigidity, tremor ratings in PD patients do not correlate with dopaminergic imaging measures of presynaptic nigrostriatal dysfunction (Ishikawa et al., 1996; Kazumata et al., 1997; Benamer et al., 2003). These findings accord with experimental animal studies (Poirier et al., 1966; Pechadre et al., 1976; Ohye et al., 1988) that have associated parkinsonian-like tremor with combined lesions of nigrostriatal dopaminergic projections and cerebello-rubral outflow pathways. Thus, nigrostriatal dopamine loss appears to be necessary but not sufficient for the development of PD tremor.

Characterization of the PD Tremor Network

In the present study, the PDTP topography was characterized by significant metabolic contributions from the cerebellum and from the primary motor cortex and striatum. The stability of this regional pattern was verified using non-parametric resampling methods (Suckling and Bullmore, 2004). Moreover, network activity values proved to have excellent within-subject reproducibility in a prospective test-retest validation sample (cf. Ma et al., 2007; Huang et al., 2007a). Perhaps most pertinent was the observation in the Vim DBS derivation cohort that baseline PDTP expression correlated with individual differences in tremor amplitude measured concurrently in the absence of stimulation. This suggests that PDTP expression is directly linked to tremor and is not indicative of stimulation per se. To substantiate these findings, PDTP expression was prospectively quantified in an independent PD patient sample and assessed the relationship between this network measure and UPDRS subscale ratings for akinesia-rigidity and tremor. Indeed, the resulting PDTP scores proved to correlate strongly with the latter but not with the former. By contrast, PDRP ratings in this cohort did not correlate with tremor ratings. Further evidence of the specificity of PDTP expression for tremor was provided by the ECD SPECT data which verified the presence of significant pattern elevation in tremor predominant patients. As with the PDRP and PDCP topographies (Ma and Eidelberg, 2007; Hirano et al., 2008), PDTP scores measured in the off-state cerebral blood flow scans are coupled to the corresponding network values measured in scans of glucose metabolism acquired in the same subjects (data not shown). It is therefore not surprising that PDTP expression could be successfully quantified in ECD SPECT perfusion scans (cf. Eckert et al., 2007). Presumably, as shown previously (Ma et al., 2010), similar network measurements will also be accessible using arterial spin labeling (ASL) perfusion MRI techniques.

Changes in Network Activity with Disease Progression and Treatment

It was also found that longitudinal changes in PDTP expression were sensitive to symptom progression. Indeed, in a previously reported early stage PD cohort who underwent longitudinal FDG PET imaging (Huang et al., 2007b; Tang et al., 2010), PDTP expression increased over time, but at a significantly slower rate than for the concurrent PDRP measurements. The progression of PDTP activity paralleled the slow rate of change in tremor ratings over the four years of observation. By contrast, the faster longitudinal increase in PDRP activity comports with the more rapid deterioration in akinesia-rigidity reported in this disease (Louis et al., 1999). The distinct time courses of PDTP and PDRP progression lend further credence to the notion that discrete pathophysiological mechanisms underlie PD tremor and the other motor manifestations of the disorder.

To determine whether and to what degree the PDTP network can be modulated by treatment, two DBS procedures known to alleviate parkinsonian tremor (Machado et al., 2006; Blahak et al., 2007) were contrasted. Improvement in tremor ratings (Table 1) was greater following Vim as compared to STN stimulation (p<0.05), which accords with concurrent treatment-mediated changes in PDTP activity measured in the same subjects. That said, consistent with the reported efficacy of Vim stimulation for parkinsonian tremor (Lyons et al., 2001; Rehncrona et al., 2003), the difference in clinical response across interventions may in part be attributed to baseline effects. Thus, the current findings do not permit a definitive statement to be made regarding the relative utility of one or the other DBS targets for the relief of PD tremor. Nonetheless, the observation that Vim stimulation gives rise to marginal improvement in akinesia-rigidity and only a modest degree of PDRP modulation underscores the specificity of this intervention for tremor pathways. By contrast, the mechanisms underlying the effects of STN stimulation on tremor are less clear and have been related to activation of the surrounding white matter, i.e., the fields of Forel, the prelemniscal radiation, and the zona incerta (see e.g., Herzog et al., 2007). It is important to consider the possibility that PDTP modulation with STN stimulation is mediated by antidromic effects on the primary motor cortex through the hyperdirect pathway (Nambu, 2004). Indeed, voxel-wise conjunction analysis disclosed shared metabolic reductions in this region during stimulation, suggesting that it may be a common final pathway for the antitremor effects observed with both Vim and STN DBS. It is conceivable that this “back door” approach to the PDTP circuit is associated with weaker network effects than the direct depolarization of thalamic cell bodies by Vim DBS. Importantly, STN DBS also affects the activity of subthalamic projections to the internal globus pallidus (GPi), thereby reducing inhibitory pallido-thalamic output and concomitantly the activity of the PDRP network (Lin et al., 2008; cf. Asanuma et al., 2006; Pourfar et al., 2009). On this basis, it is not surprising that by modulating the activity of both PDRP and PDTP, STN stimulation can improve both akinesia-rigidity and tremor in PD patients. Moreover, the cerebellum has recently been found to receive substantial disynaptic projections from the STN (Bostan et al., 2010). This pathway may represent an additional means by which STN interventions can influence these two PD-related metabolic networks.

Anatomical and Functional Basis for the PD Tremor Network

The akinetic-rigid manifestations of PD have been associated with discrete functional abnormalities of cortico-striatopallido-thalamocortical (CSPTC) motor circuits (DeLong and Wichmann, 2007). These changes, however, do not readily account for other disease manifestations such as tremor (Zaidel et al., 2009). Indeed, tremor generation has been linked to abnormal activity in cerebello-thalamo-cortical (CbTC) pathways (Volkmann et al., 1996; Timmermann et al., 2003), and the role of the basal ganglia in mediating this symptom has remained the subject of debate (see Deuschl et al., 2000; Timmermann et al., 2007 for review). Indeed, prior imaging studies have shown that both lesioning and high frequency stimulation of the Vim thalamic nucleus results in localized reductions in neural activity in the primary motor cortex and the anterior cerebellum (Baron et al., 1992; Deiber et al., 1993; Boecker et al., 1997; Wielepp et al., 2001; Fukuda et al., 2004). In keeping with these findings, magnetoencephalography (MEG) studies with EMG back-averaging disclosed a tremor-coherent oscillatory network involving the primary motor cortex, thalamus, and cerebellum, which also contribute significantly to the PDTP metabolic topography. Interestingly, the PDTP metabolic topography also included significant contributions from the striatum, albeit of lower magnitude than the other nodes of this network. In the primate, the striatum receives cerebellar output via the ventrolateral and intralaminar thalamic nuclear groups (Hoshi et al., 2005), and metabolic activity in the putamen was found to correlate with tremor ratings in another FDG PET study (Lozza et al., 2004). In aggregate, findings from both MEG and PET suggest that the regional nodes of the PD tremor network are defined by abnormal synchronization of firing, leading to localized increases in synaptic activity and concomitant elevations in glucose metabolism. While the observed tremor-related changes are most prominent in the primary motor cortex and cerebellum, these PDTP regions interconnect through the Vim thalamus and putamen, thus describing a distinct large-scale metabolic network associated with this disease manifestation. The thalamus itself did not contribute to the PDTP regional topography.

Interestingly, a post-hoc volume-of-interest (VOI) analysis did reveal a stimulation-related (ON>OFF) increase in Vim thalamic metabolic activity (p<0.02, paired Student's t-test). This is consistent with prior reports of increased regional cerebral blood flow and metabolism at the Vim electrode insertion site (Rezai et al., 1999; Perlmutter et al., 2002; Haslinger et al., 2003; Fukuda et al., 2004). Nonetheless, Vim thalamic metabolic activity in the DBS cohort did not differ from normal control values in either stimulation condition (OFF: p=0.74; ON: p=0.58), and failed to correlate with UPDRS tremor subscale ratings (n=41; r=0.21, p=0.19) in prospective scan data. Moreover, the stimulation-mediated changes observed in the Vim thalamus did not correlate (r=0.18, p=0.65) with concurrently measured PDTP changes. These findings suggest that the regional thalamic changes occurring with stimulation are not critical to the tremor-related spatial covariance pattern identified with OrT CVA. It is likely that the increases in thalamic blood flow and metabolic activity observed with Vim stimulation reflect direct effects on local cell membrane potentials at the electrode tip, rather than functional effects at downstream thalamic output pathways. By contrast, the ventrolateral thalamus, particularly the pallido-receptive Voa/Vop nuclei, contributes functionally to the PDRP topography (Lin et al., 2008; Eidelberg et al., 1997). Indeed, the observed topographic difference between PDTP and PDRP is compatible to the known segregation of cerebellar- and pallidal-receiving circuits at the thalamic level (Middleton and Strick, 2000).

In addition, network-related activation of the sensorimotor cortex and cerebellum is a known accompaniment of normal movement. Nevertheless, no spatial homology was found between the PDTP topography and the previously characterized normal movement-related activation pattern (NMRP) (Carbon et al., 2010). These results suggest that the PDTP is a truly abnormal metabolic network and cannot be construed simply as an overactive fragment of the normal motor circuit. Similarly, partial coherence analysis of MEG data from patients with PD tremor suggests that the tremor-related regional changes are not the consequence of increased somatosensory input from rhythmic muscle activity (Timmermann et al., 2003).

### Experimental Results II

### Introduction

Huntington's disease (HD) has been the focus of therapeutic initiatives to slow or arrest the disease in presymptomatic mutation carriers. Huntington's disease (HD) is an autosomal dominant neurodegenerative disorder characterized by progressive impairments in motor, cognitive, and affective functions. The disorder is caused by a fully penetrant mutant gene with an unstable CAG expansion located on the short arm of chromosome 4 encoding the neurotoxic Huntingtin protein. Carriers of this mutation can be identified many years before clinical diagnosis, making it possible in principle as well as economically sound to devote resources to developing treatments for delaying or preventing the onset of symptoms. However, the objective assessment of therapies designed to modify the course of HD depends on the availability of sensitive and reliable biomarkers of disease progression in the preclinical and early symptomatic stages of the illness. While clinical rating scales such as the Unified Huntington's Disease Rating Scale (UHDRS) are currently the “gold standard” for assessing HD severity, such measures are insensitive to disease progression in premanifest subjects or in individuals at or near the onset of symptoms. Alternatively, imaging tools such as [¹¹C] raclopride (RAC) positron emission tomography (PET) to measure reductions in binding to caudate and putamen dopamine D2 neuroreceptors and volumetric MRI to assess tissue loss in these brain areas (J. S. Paulsen, 2009) have been used to estimate the rate of disease progression in “at risk” individuals. Whereas these methods provide in vivo measurements of the rate of striatal neurodegeneration in HD, regional measurements provide scant information concerning the broader functional topography of the disease process (e.g., (D. Eidelberg et al. (2011), M. Esmaeilzadeh et al. 2010)). In fact, little is known about the time course of the spatially distributed changes in brain function that take place during the premanifest and early symptomatic phases of the illness.

Network analysis has provided a robust means of identifying specific patterns of abnormal regional connectivity in functional brain images from individuals with neurodegenerative disorders (D. Eidelberg, 2009), as well as from preclinical subjects with prodromal disease (C. C. Tang et al., 2010; , A. Feigin, 2007) and a form of such is employed here.

### Materials and Methods

Subjects

Twelve premanifest Huntington's disease (HD) mutation carriers (male/female: 5/7; baseline age: 46.8±11.0 years (mean±SD), range 25-62 years; CAG repeat length: 41.6±1.7, range 39-45; predicted years-to-onset: 10.3±8.6, range 1-25 years) underwent longitudinal imaging with [¹⁸F]-fluorodeoxyglucose (FDG) and [¹¹C]-raclopride (RAC) positron emission tomography (PET), structural magnetic resonance imaging (MRI) and serial clinical ratings including the Unified Huntington's Disease Rating Scale (UHDRS) (Mov Disord 11, 136 (March, 1996)). Baseline imaging and clinical assessments were performed on all subjects (n=12) in this group (HD1) and were repeated after 1.6±0.1 (n=12), 3.7±0.3 (n=10), and 7.2±0.4 (n=9) years (mean±SD). Mean total motor UHDRS ratings for this longitudinal premanifest HD cohort are presented in Table 4.

TABLE 4
HD mutation carriers: UHDRS motor ratings and measurements
of caudate/putamen D₂ binding and tissue volume

| | HD1 (longitudinal cohort) | | | | HD2 | |
|---|---|---|---|---|---|---|
| | Baseline | 1.5 years | 4 years | 7 years | (symptomatic) | |
| | (n = 12) | (n = 12) | (n = 10) | (n = 9) | (n = 5) | HC* |
| UHDRS (motor) | | | | | | |
| Phenoconverters | 23.8 (9.8)^{†} | 22.7 (11.0) | 27.0 (10.9) | 33.3 (9.2) | 42.8 (4.4) | N/A |
| Non-phenoconverters | 2.5 (2.5) | 5.5 (6.7) | 2.2 (1.0) | 2.0 (1.6) | | |
| Total | 9.6 (11.8) | 10.2 (10.9) | 12.1 (14.3) | 15.9 (17.4) | | |
| Caudate D₂ binding | | | | | | |
| Phenoconverters | 0.92 (0.42), | 0.95 (0.22), | 0.79 (0.28), | 0.71 (0.19), | 0.72 (0.20), | 2.09 (0.43), |
| | 43.8^{‡} | 45.2 | 37.6 | 33.7 | 34.6 | 100.0 |
| Non-phenoconverters | 1.50 (0.27), | 1.39 (0.30), | 1.31 (0.38), | 1.28 (0.04), | | |
| | 71.9 | 66.4 | 62.6 | 61.2 | | |
| Total | 1.34 (0.40), | 1.23 (0.34), | 1.10 (0.43), | 1.06 (0.32), | | |
| | 64.3 | 58.7 | 52.6 | 50.9 | | |
| Putamen D₂ binding | | | | | | |
| Phenoconverters | 1.01 (0.25), | 1.02 (0.11), | 0.87 (0.13), | 0.79 (0.11), | 0.80 (0.22), | 2.07 (0.39), |
| | 48.9 | 49.4 | 42.1 | 38.1 | 38.8 | 100.0 |
| Non-phenoconverters | 1.50 (0.29), | 1.36 (0.28), | 1.29 (0.27), | 1.26 (0.08), | | |
| | 72.7 | 65.7 | 62.1 | 61.0 | | |
| Total | 1.37 (0.35), | 1.24 (0.28), | 1.12 (0.30), | 1.08 (0.26), | | |
| | 66.2 | 59.7 | 54.1 | 52.4 | | |
| Caudate volume | | | | | | |
| Phenoconverters | 1.60 (0.74), | 1.48 (0.72), | 1.44 (0.74), | 1.12 (0.41), | 1.40 (0.29), | 2.51 (0.50), |
| | 63.8 | 59.0 | 57.2 | 44.7 | 55.6 | 100.0 |
| Non-phenoconverters | 2.16 (0.46), | 2.06 (0.47), | 2.11 (0.49), | 1.97 (0.31), | | |
| | 85.9 | 82.1 | 84.1 | 78.2 | | |
| Total | 1.97 (0.60), | 1.85 (0.61), | 1.87 (0.65), | 1.59 (0.56), | | |
| | 78.5 | 73.7 | 74.3 | 63.3 | | |
| Putamen volume | | | | | | |
| Phenoconverters | 2.27 (0.56), | 2.27 (0.64), | 2.14 (0.60), | 1.88 (0.58), | 2.00 (0.48), | 3.35 (0.64), |
| | 67.7 | 67.8 | 63.7 | 56.0 | 59.6 | 100.0 |
| Non-phenoconverters | 3.27 (0.73), | 3.03 (0.75), | 2.92 (0.66), | 2.93 (0.38), | | |
| | 97.6 | 90.5 | 86.9 | 87.3 | | |
| Total | 2.94 (0.82), | 2.76 (0.78), | 2.63 (0.73), | 2.46 (0.71), | | |
| | 87.6 | 82.2 | 78.5 | 73.4 | | |

^{†}Mean (SD).
^{‡}% of the normal mean.
UHDRS = Unified Huntington's Disease Rating Scale; HD = Huntington's disease; HC = healthy control.
*n = 12 for RAC PET; n = 18 for MRI.

At baseline, none of the 12 gene carriers were judged to have a clinically definite diagnosis by a movement disorders specialist with expertise in HD who was blind to the imaging data. However, during the course of the study, four of the initially premanifest gene carriers phenoconverted, i.e., were given a clinical diagnosis of definite HD. Two of these subjects were diagnosed with HD at 1.5 years, and two others at 4 years. In this study, the four premanifest subjects who subsequently developed sufficient clinical manifestations for diagnosis were referred to as “phenoconverters”; the remaining eight premanifest subjects were referred to as “non-phenoconverters”.

Two groups of healthy volunteer subjects served as controls for the network assessments. The first healthy control group (HC1) consisted of 12 normal subjects (male/female: 6/6; age 40.8±14.7, range 27-66 years) who underwent FDG PET at a single time point for comparison with the baseline scans of the premanifest HD gene carriers (A. Feigin (2007)). These scans were used to standardize the subject scores for the HD progression pattern identified by network analysis of the longitudinal FDG PET data (see below). The second group of healthy control (HC2) subjects consisted of 20 subsequent normal volunteers (male/female: 10/10; age 47.7±13.5 years, range 21-68 years) who also underwent FDG PET imaging at a single time point. These scans were used as part of prospective network validation. Separate age-matched groups of healthy subjects served as controls for the RAC PET (n=12, male/female: 5/7, age 42.5±15.6, range 22-64 years) and the MRI (n=18, male/female: 7/11, age 39.8±15.1, range 22-66 years) studies.

Once identified, the HD progression pattern was validated in a separate testing group (HD2) of 14 gene carriers comprised of nine premanifest HD subjects (male/female: 3/6; age 38.5±12.3, range 20-55 years; CAG repeat length: 41.4±1.4, range 40-44; predicted years-to-onset: 13.8±5.9, range 7-21 years) and five early symptomatic HD patients (male/female: 0/5; age: 53.8±6.3 years, range 43-59 years; UHDRS motor ratings: 42.8±4.4 years, range 38-50) who were scanned once between two and four years (mean 3.0±0.71 years) after clinical diagnosis. Subject scores for the HD progression pattern were quantified in the FDG PET scans from this group of gene carriers (HD2) and from the second healthy control group (HC2) on a prospective single case basis. For validation, the test-retest reliability of the pattern expression was assessed in repeat FDG PET scans (mean interval 24.2±10.5 days) acquired in the nine premanifest subjects included in the HD2 prospective testing group. The test-retest studies were performed at four PET sites (Site 1: North Shore University Hospital; Site 2: Indiana University; Site 3: University of Iowa; Site 4: University of Toronto) as part of the PREDICT-HD consortium.

Lastly, to confirm the estimate of the rate of network progression in preclinical HD, we measured pattern expression in FDG PET data from an independent longitudinal cohort of premanifest HD carriers. This cohort was studied at the University Medical Center, Groningen, Netherlands as described elsewhere (J. C. van Oostrom et al. (2005); J. C. van Oostrom et al. (2009)). It was comprised of 21 premanifest HD mutation carriers (male/female: 9/12; age: 40.3±6.8 years, range 29-57 years; CAG repeat length: 42.9±2.3, range 39-47; predicted years-to-onset: 11.7±6.5, range 1-25 years) who were scanned at baseline and again 2.3±0.3 years later.

Ethical permission for these studies was obtained from the Institutional Review Board of North Shore University Hospital and University Medical Center Groningen. Written informed consent was obtained from each subject following detailed explanation of the procedures.

### Imaging Procedures

Positron Emission Tomography

Members of the HD1 longitudinal cohort of premanifest mutation carriers underwent FDG and RAC PET at baseline and at the subsequent time points. At each visit, scanning with the two radiotracers was performed over a 2-day period using the GE Advance tomograph (General Electric Medical Systems, Milwaukee, Wis.) at North Shore University Hospital (5). In the cross-sectional HD2 gene-positive testing group, the five early symptomatic HD patients and one of the nine premanifest subjects participating in the test-retest study were scanned on the GE Advance device at North Shore University Hospital. The remaining eight premanifest members of the HD2 group underwent test-retest studies on the Siemens HR+ scanners at Indiana University (n=2) and the University of Iowa (n=3), and on the Siemens HRRT tomograph at the University of Toronto (n=3). The 21 premanifest HD subjects in the prospective longitudinal cohort used to confirm the estimate of the network progression rate were scanned with FDG PET using the Siemens ECAT Exact HR+ scanner (Siemens Erlangen, Germany) at University Medical Center Groningen, Netherlands.

For FDG PET, a 10 min scan was acquired in three-dimensional (3D) mode beginning 35 min after the intravenous injection of 5 mCi of radiotracer. The studies were performed after an overnight fast, with the subjects' eyes open in a dimly lit room and with minimal auditory stimulation. Longitudinal scans from each premanifest subject were realigned and spatially normalized to a standard Talairach-based FDG PET template, and smoothed with an isotropic Gaussian kernel (10 mm) in all directions to improve the signal-to-noise ratio (A. Feigin (2007); C. Huang (2007)). The scans from the prospective HD and healthy control groups were individually normalized and smoothed.

For RAC PET, the subjects received 15 mCi of radiotracer by intravenous injection and dynamic images were acquired over 70 minutes (7×10 minutes), as described previously (2). The individual frames were spatially realigned to compensate for potential movement during scanning and were transformed into standard Talairach brain space. All normalized images involving the striatum were integrated into a single slice. Regions-of-interest (ROIs) were defined anatomically on each image with reference to a template in standard space using an automated procedure. Specifically, ROIs were placed bilaterally on the caudate nucleus, putamen and occipital regions of each scan, blind to subject identity, mutation status (gene positive or negative), clinical status (premanifest or symptomatic), and time point. D₂ receptor binding affinity was separately estimated for the caudate and putamen by computing the striatal-occipital ratio (ROI/occiptal-1) between 50 and 60 minutes post-injection. The same set of standardized ROIs was used for the longitudinal scans from the premanifest subjects, and for the prospective HD and healthy control scans. For each ROI, left and right values from the gene carriers were averaged and compared with the corresponding control values.

Magnetic Resonance Imaging

Subjects in the HD1 longitudinal gene-positive HD cohort were scanned on the 1.5T GE Signa Echo Speed scanner at North Shore University Hospital. T1-weighted images were acquired with a 3D spoiled gradient recall sequence (TE=5 ms, TR=24 ms, flip angle=20°), with matrix size 256×256×124 giving resolution of 1-1.5 mm in the transverse and axial planes. These images were used to quantify caudate and putamen volume at each longitudinal time point in the premanifest cohort. Analogous volumetric measurements were conducted in the symptomatic members of the HD2 testing cohort and in healthy control scans. For the longitudinal cohort, follow-up images were aligned to the baseline scans using a least-squares approach and a six-parameter (rigid-body) spatial transformation to minimize repositioning errors across different time series.

To assess changes in striatal volume over time, manual segmentation was performed to measure caudate and putamen volume in the original MRI scans. This was performed with MRIcro software (available at: http://www.cabiatl.com/mricro/mricro/index.html) utilizing the aligned MRI scans in native space. In each MRI scan, contours of caudate and putamen were separately outlined in the axial slices in which these structures were clearly visible. For each region, volumes on the left and right sides of the brain were separately calculated across all slices and then averaged across hemispheres. Caudate and putamen volumes were measured for the premanifest subjects at all four longitudinal time points and for the prospective HD and control groups at a single time point.

### Network Analysis

Pattern Identification

A within-group network modeling approach was used to identify patterns of regional functional connectivity in premanifest HD mutation carriers that increase monotonically in their expression with disease progression. The computational algorithm, termed Ordinal Trends (OrT) Canonical Variates Analysis (CVA) (software available at groups.google.com/group/gcva) has been described in detail elsewhere (C. Habeck (2005); J. R. Moeller (2006)). Based on supervised PCA, this mathematical-statistical model searches for specific patterns of functional connectivity (i.e., large scale brain networks) in serial imaging data acquired over multiple ordered experimental conditions. OrT/CVA uses a specially formulated transformation of the voxel×condition×subject data matrix prior to single value decomposition. In this way, the analysis seeks to detect a specific class of spatial covariance patterns characterized by monotonically increasing (or decreasing) pattern expression over time on an individual case basis, while the functional relationships between the brain regions comprising the pattern topography remain constant. In other words, the model identifies significant functional brain networks that exhibit an ordinal trend in subject activity, i.e., a consistent increase (or decrease) in pattern expression in all or most members of the derivation cohort. In this regard, OrT/CVA differs from typical mass-univariate voxel-based analyses in that it requires network activity to change consistently on a subject-by-subject basis, rather than on a group mean basis. Moreover, OrT/CVA is guided solely by the design variables, which in this analysis encode the temporal ordering of the scans for each subject. Importantly, the pattern identification procedure that we performed did not require or utilize knowledge of experimental predictor variables or demographic factors such as CAG repeat length, subject age, or the number of years estimated to remain until clinical onset.

In addition to the identification of relevant spatial covariance pattern(s) in the longitudinal imaging data, OrT/CVA quantifies the expression of the corresponding pattern(s) in each subject and condition in the derivation cohort and in prospective testing populations. The significance of candidate progression topographies is determined by non-parametric inferential tests (C. Habeck (2010)). Permutation tests of the associated principal component (PC) scalars (subject scores) are performed to assess the possibility that the changes in pattern expression observed across subjects/conditions (i.e., the ordinal trend) in the derivation data set had occurred by chance. The voxel loadings (region weights) on the covariance pattern specify the spatial topography of the network, reflecting local contributions to its overall activity. The reliability of each voxel weight can be estimated and mapped using bootstrap procedures (B. Efron et al. (1994)).

In the current study, we posited that as a fully penetrant dominantly inherited neurodegenerative disorder, preclinical HD is likely to exhibit consistent subject-by-subject longitudinal changes at the network level conforming to an ordinal trend. To test this hypothesis, we used OrT/CVA to search for a significant HD progression covariance pattern in the longitudinal metabolic imaging data of the premanifest mutation carriers. The HD progression pattern was sought among the linearly independent (orthogonal) principle component (PC) patterns that resulted from the analysis of the metabolic imaging data from the first three experimental time points. Pattern selection was selected based upon the following criteria: (1) the search for appropriate patterns was limited to the PCs with the highest eigenvalues; and (2) subject scores for these PCs were entered singly and in all possible combinations to achieve the maximal separation between subject contrast scores (C. Habeck et al. (2005)). The Akaike information criterion (AIC) (K. P. Burnham (2002)) was used to specify the optimal linear combination of subject contrast scores, i.e. the set of PCs with the best bias-variance trade off (C. Habeck et al. (2005)).

The resulting progression pattern was considered significant if the associated subject scores exhibited a monotonically increasing trend over time that differed from chance at p<0.05, permutation test with 1,000 iterations). The coefficients on the subject scores for the regression model were applied to the respective PCs to yield the corresponding spatial covariance topography. The reliability of the voxel weights on the resulting pattern was tested using a bootstrap resampling procedure with 1,000 iterations. The threshold for voxel weight reliability was set at |ICV|=1.96, corresponding to p<0.05, two-tailed.

Pattern Validation

Following the identification of a significant progression pattern in the three time point longitudinal premanifest derivation sample, we quantified its expression on a single subject/scan basis in several independent prospective testing datasets: (1) the fourth time point (i.e., seven-year follow-up) scans of the HD1 longitudinal premanifest cohort (n=9); (2) the scans from the prospective HD2 cross-sectional testing group (n=14) including the test-retest scans from the nine premanifest members of this group (3) the scans from the original (n=12) and subsequent (n=20) healthy control groups (HC1 and HC2, respectively); and (4) the scans from the second longitudinal cohort of premanifest carriers (n=21), which were used to confirm the estimated rate of network progression. Pattern expression values for all the scans (derivation and validation) were standardized by z-transformation with respect to the HC1 control group, such that these normatives had a mean subject score of zero and a standard derivation of one. All network quantification procedures were performed blind to time point, subject, years-to-onset, clinical diagnosis, and UHDRS ratings.

Regional Analysis

In addition to the network analysis, we measured the time course of regional metabolic activity at the major nodes of the HD progression pattern. Identical spherical volumes-of-interest (VOIs) (radius=4 mm) were centered on the peak voxel of each network region in standard space. To reduce intersubject variability, the measured activity in each VOI was ratio normalized by the global metabolic rate measured in the corresponding scan. The resulting regional values from the HD gene carriers were plotted and displayed with reference to the HC1 control cohort.

Effects of Volume Loss on Network Activity

A segmentation algorithm (Voxel Based Morphometry Toolbox available at http://dbm.neuro.uni-jena.de/vbm/) was used in standard space to delineate gray matter voxels in each MRI scan (S. S. Keller (2004); H. H. Ruocco (2008)). The resulting voxel-based morphometric (VBM) scans from the first three longitudinal time points of the HD1 cohort (corresponding to the metabolic imaging data points used for pattern identification) were interrogated for regions with significant loss of tissue volume over time using statistical parametric mapping (SPM 5, Institute of Neurology, London, UK). A flexible factorial repeated measures design was used, and the resulting SPM {t} maps were thresholded at p<0.001, with a false discovery rate (FDR) correction at p<0.05.

The resulting three-dimensional (3D) brain map of the regions with significant gray matter volume loss was used to construct a hypothesis-testing mask with which to determine whether progressive atrophy influenced the rate of pattern progression. To this end, the metabolic images from the three longitudinal time points were further analyzed by dividing each brain volume into two subspaces: one inside and the other outside the pre-specified mask. In each FDG PET scan, pattern expression was quantified separately for the two subspaces. Pattern expression measured inside the mask was assumed to relate closely to progressive regional brain atrophy. By contrast, pattern expression outside the mask was considered to be less related to concurrent changes in tissue volume. This subspace was hypothesized to represent the “functional” component of progression-related network activity. The changes in pattern activity measured in the two subspaces were used to estimate the rate of network progression with and without the contribution of concurrent volume loss (see below).

Statistical Analysis

For the test-retest validation studies, the reproducibility of prospectively measured network values (subject scores) in individual subjects was assessed by computing the intraclass correlation coefficient (Y. Ma (2007)). The rate of metabolic network progression in the original longitudinal premanifest HD cohort was estimated from the whole brain network values and the corresponding predicted years-to-onset at the four longitudinal time points. This was accomplished using individual growth models (J. D. Singer et al. (2007)). In addition, rates of progression were determined for the network values computed in each of the two metabolic image subspaces, i.e., inside and outside the volume loss mask (see above). A second longitudinal cohort of premanifest carriers was used to confirm the original estimate of the network progression rate. In this group, the prospectively computed network values for each subject/time point and the respective years-to-onset measures were similarly analyzed using IGM to calculate a corresponding rate of network progression.

Progression rates were additionally calculated from the longitudinal caudate and putamen D₂ receptor binding data (RAC PET) and the corresponding tissue volume measures (volumetric MRI) obtained in the same subjects at the four time points. To compare the latter rates with those determined at the network level, the striatal progression indices were z-scored with respect to the corresponding control mean values and plotted against years-to-onset. Because these measures declined over time while pattern scores increased, the corresponding regression lines were reversed (“flipped”) so that the slopes of all the progression parameters were positive.

For each progression measure, the longitudinal scan data for all the premanifest subjects and time points in the HD1 cohort were entered into individual growth curve models, including the cases with incomplete data. The rate of progression for each imaging descriptor was estimated as a continuous function of “disease time”, defined as the number of years-to-onset at each experimental time point. For the premanifest subjects who became symptomatic during the study, the predicted years-to-onset was replaced by the actual number of years before or after the time of clinical diagnosis. Longitudinal trajectories were evaluated with linear (years-to-onset) and curvilinear (years-to-onset² or In (years-to-onset)) models. For each measure, the model with the best fit to the data, i.e., that with the lowest AIC value, was selected. Unless the non-linear fit proved superior, the estimation of the progression parameters relied on linear growth models. Individual growth models were also used for the direct comparison of the progression rates estimated in the longitudinal HD1 premanifest cohort based upon the different imaging measures.

In addition to estimating the annual rate of change (i.e., the slope) for each measure, the model provided the estimated value for each imaging measure that was associated with phenoconversion (i.e., the Y-axis intercept, when years-to-onset equaled zero). Based on the model, we also calculated when each measure began to deviate from the normal mean (i.e. the X-axis intercept, when the z-scored imaging measure equaled zero) and when abnormal levels were reached (i.e., exceeding 2 SD above or below the normal mean value). All statistical analyses were performed using SAS 9.1 (SAS Institute Inc.) and the significance level was set at p<0.05.

### Results

A cohort of premanifest HD subjects underwent longitudinal metabolic imaging at four discrete time points over a seven-year period. By identifying and validating a distinct HD progression-related network in premanifest mutation carriers and quantifying changes in its activity over time, it was possible to measure the rate of the disease process at the systems level. Moreover, by additionally scanning the subjects with both [¹¹C] raclopride (RAC) PET and structural MRI at each time point, we assessed concurrent declines in caudate/putamen D₂-receptor binding and tissue volume, two regional indicators of preclinical HD progression.

The HD Progression Pattern

Pattern Identification:

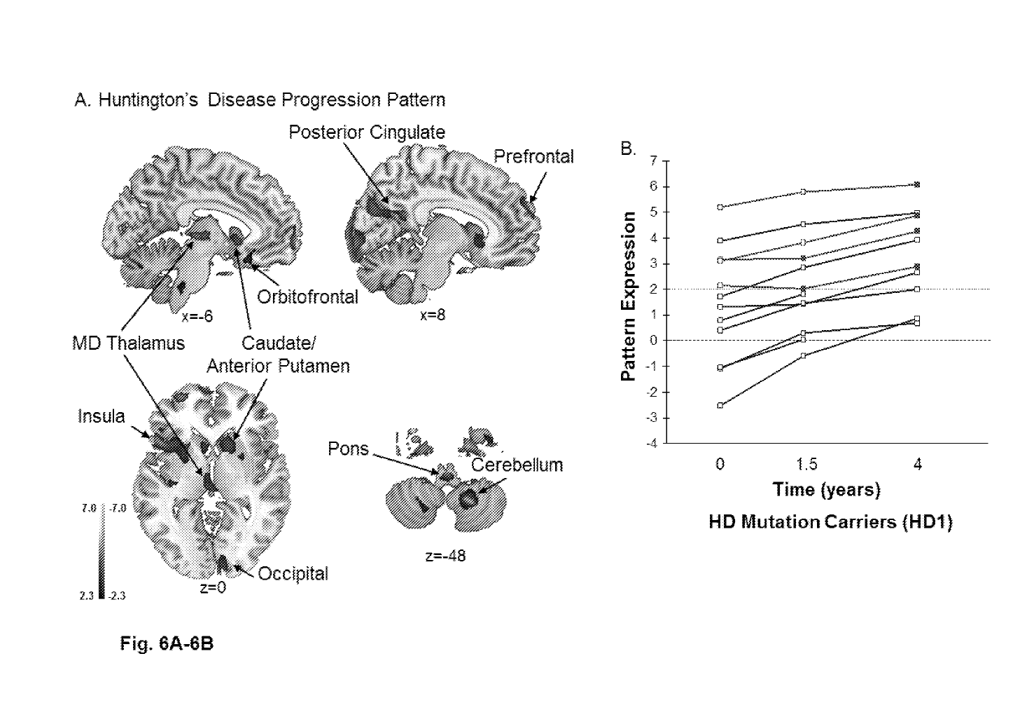

To identify a spatial covariance pattern specifically associated with HD progression in the preclinical period, longitudinal metabolic imaging data was examined from a group of 12 premanifest mutation carriers designated HD1 (age: 46.8±11.0 years (mean±SD), range 25-62 years; CAG repeat length: 41.6±1.7, range 39-45; predicted years-to-onset: 10.3±8.6, range 1-25 years). A network modeling algorithm (J. R. Moeller, (2006); C. Habeck et al. (2005)) was employed to detect patterns of regional functional connectivity with monotonically changing expression over time. Significant spatial covariance patterns identified in the data using this approach exhibited an “ordinal trend” in subject activity, i.e., a consistent increase (or decrease) in pattern expression over time in all or most of subjects, even as the functional relationships between the individual brain regions remained constant. Indeed, analysis of the longitudinal scan data acquired at baseline, 1.5 and 4 years revealed a significant progression-related metabolic covariance pattern (FIG. 6A) that accounted for 9.7% of the overall voxel×subject×time variance. Without exception, all of the premanifest mutation carriers exhibited a monotonic increase (p<0.001, permutation test) in pattern expression during this time period (FIG. 6B). The metabolic network was characterized by a distinct spatial topography (Table 4), with progressively declining regional activity in the striatum, thalamus, insula and posterior cingulate area, and in the prefrontal and occipital cortex. These changes covaried with increasing regional activity in the cerebellum, pons, hippocampus, and orbitofrontal cortex. The voxel weights (loadings) on the pattern, which define the contribution of each region to overall network activity, were found to be highly reliable on bootstrap resampling (p<0.0001, inverse coefficient of variation (ICV) range=[−6.02, 5.63]; 1,000 iterations).

Pattern Validation:

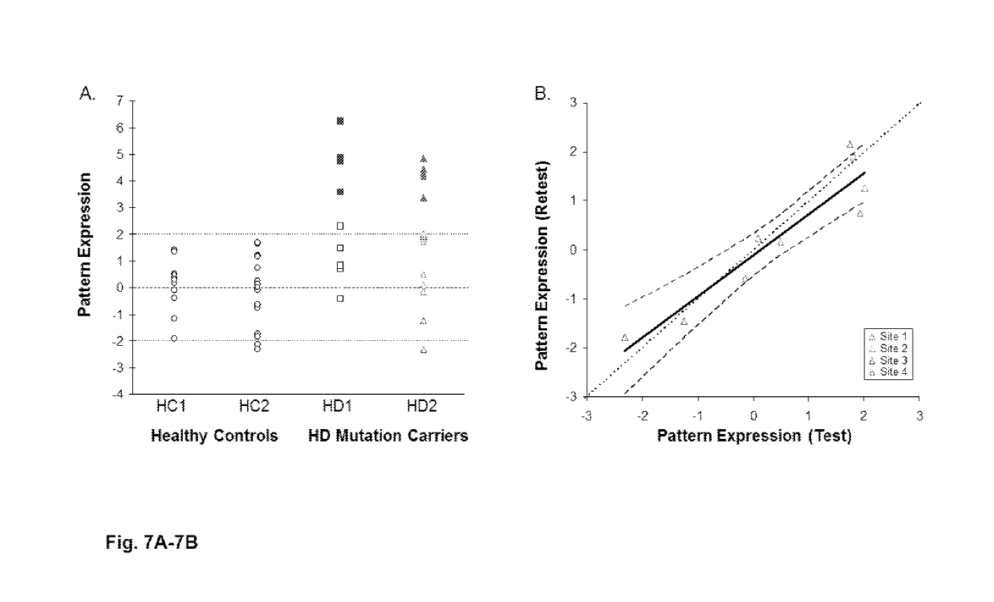

Nine members of the longitudinal HD cohort returned for final imaging assessment at seven years. By this time, four of the nine subjects had phenoconverted (i.e., developed overt, clinical manifestations of HD); the other five remained “non-phenoconverters.” Prospectively computed network activity values for the nine subjects (FIG. 7A) were increased relative to baseline (p<0.0001, paired Student's t-test). Additionally, we found that pattern expression at this time point (2.7±2.3, mean±SD) was elevated (p=0.001, Student's t-test) with respect to a group of 12 healthy control subjects (0.0±1.0) designated HC1 (age 40.8±14.7 years, range 27-66 years). Network values computed for the four phenoconverters were higher than concurrently measured values for the five non-phenoconverters (mean subject scores: 4.86 vs. 0.99).

Next, the activity of the network on a prospective single case basis was computed in two additional groups of subjects: HD2, an independent testing cohort comprised of 14 additional HD gene carriers (nine premanifest and five early symptomatic subjects; age 38.5±12.3, range 20-55 years; CAG repeat length: 41.4±1.4, range 40-44; predicted years-to-onset: 13.8±5.9, range 7-21 years), scanned at four separate PET sites (see Methods); and HC2, a second control group comprised of 20 healthy control subjects (age 47.7±13.5 years, range 21-68 years). Network values differed across groups (FIG. 7A; F_{(2,43)}=7.1, p<0.005; one-way ANOVA), with elevated expression in the HD2 testing cohort (1.8±2.2, mean±SD) relative to the HC1 (p<0.05, post-hoc Bonferroni test) and HC2 (−0.1±1.3, p<0.005) healthy control groups. Pattern expression computed in the HC2 testing group did not differ (p=0.99) from the HC 1 values that were used to standardize the network measurements. Network values in the five early symptomatic HD2 subjects (measured, on average, 3.0 years after clinical diagnosis) were similar to those measured in the four HD1 phenoconverters at seven years (on average, 4.5 years after clinical diagnosis). Indeed, each of these nine clinically diagnosed mutation carriers exhibited network elevations of 3 SD or more above the normal mean. Each of the nine premanifest gene-carriers in the HD2 cohort underwent repeat metabolic imaging over a three week interval. Test-retest evaluation of network expression in these individuals (FIG. 7B) revealed an excellent degree of within-subject reproducibility for this measure (Intra-class correlation coefficient (ICC)=0.96, p<0.001).

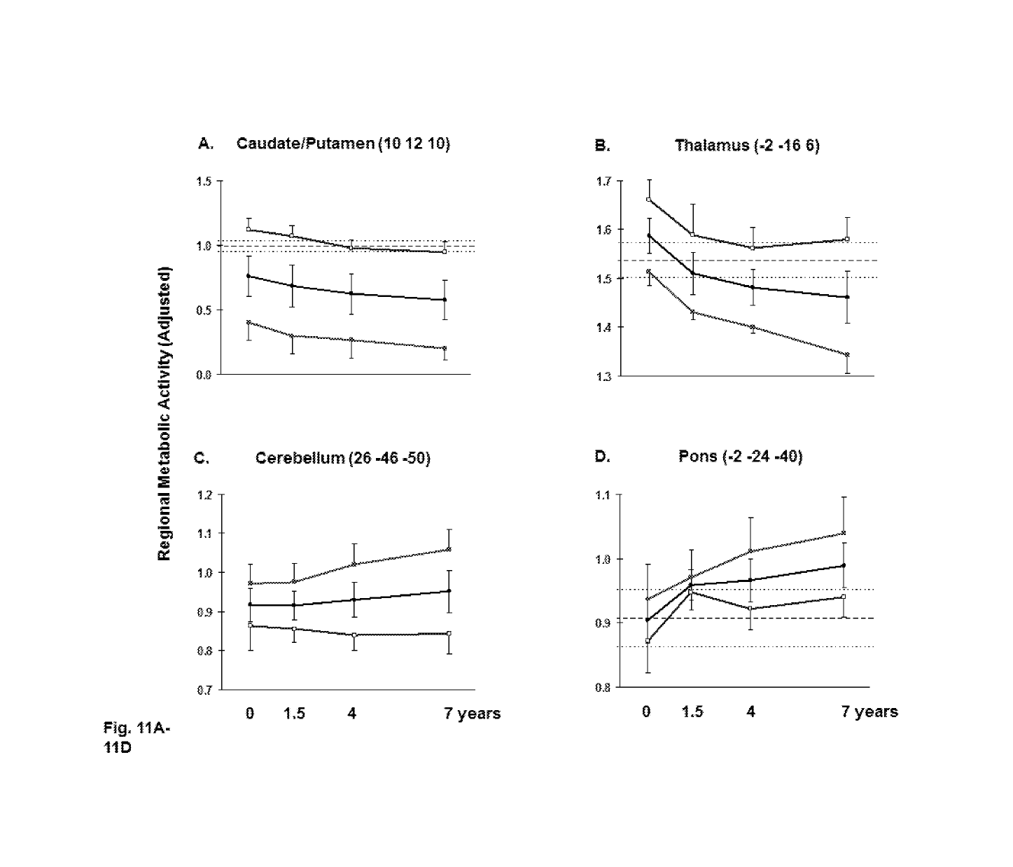

Regional Analysis. Changes in regional metabolic activity were then examined at each of the major nodes of the HD progression-related network (FIG. 11). With advancing disease, metabolic activity declined in the caudate/putamen (p<0.0001; Individual Growth Model, IGM), mediodorsal thalamus (p<0.0001), insula (p<0.0001) and posterior cingulate region (p<0.005), and in the prefrontal (p<0.005) and occipital (p<0.05) cortex. Decreasing striatal metabolism in HD is likely to reflect the effects of local volume loss as well as declining neuronal function in this brain region (E. H. Aylward et al. (1997); B. G. Jenkins et al. (2005)). Metabolic decline in the mediodorsal thalamus is consistent with loss of compensation for declining striatal function as symptoms emerge (A. Feigin et al. (2007); A. Feigin et al. (2006)). While the thalamic changes are likely to reflect functional alterations in synaptic activity with ongoing disease, volume loss in other areas in preclinical HD (H. D. Rosas et al. (2002); H. D. Rosas et al. (2004)) may in part underlie the decline in metabolic activity noted in these regions. By contrast, progressive increases in metabolic activity were noted in several brain regions in premanifest HD carriers. Such changes were evident in the cerebellum (p<0.05) and pons (p<0.01), perhaps as a metabolic prodrome for the motor manifestations of the disease which subsequently emerged in the phenoconverters. A significant longitudinal increase in regional metabolic activity was evident in the temporal cortex (BA 37/38, p<0.05), but did not reach significance at the other increasing network nodes (hippocampus: p=0.34; orbitofrontal: p=0.35; lateral occipital: p=0.08). The progressive metabolic increases observed in these regions suggest a compensatory role (A. Feigin et al. (2006)), which can be established only through longer term follow-up studies.

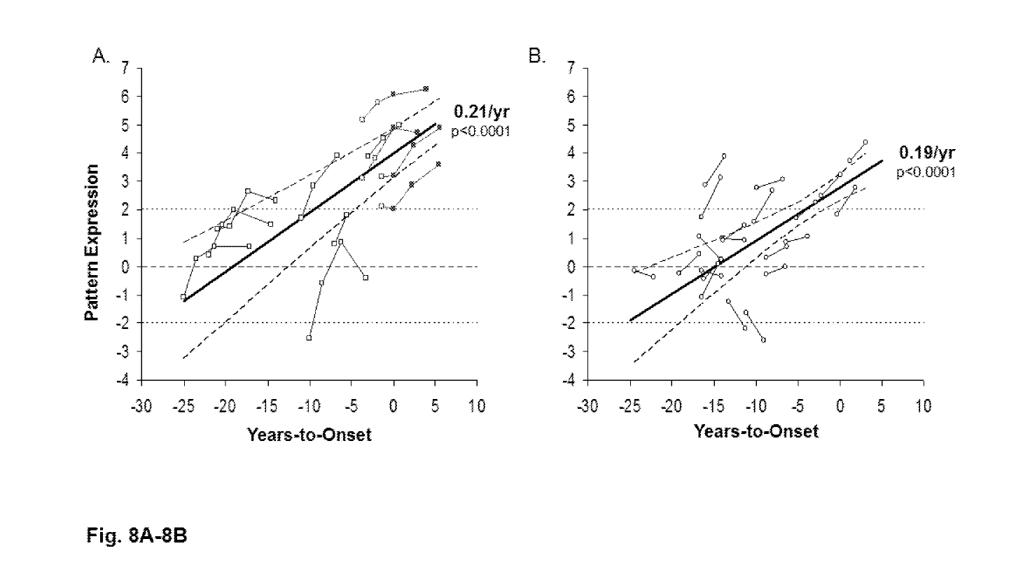

Network Activity as a Biomarker of HD Progression. To measure the rate of network progression in premanifest HD, we assessed the longitudinal changes in pattern expression that were observed as a function of “disease time,” defined in each subject/time point as the number of years remaining until the predicted time of clinical onset. (For the four phenoconverters in group HD1, we used the number of years until actual diagnosis). The data show that the increases in network activity with time are directly proportional to advancing disease expressed as declining years-to-onset (FIG. 8A). The progression rate for network activity was estimated to be 0.21/year (p<0.0001; 95% confidence interval (CI)=[0.15, 0.27], IGM).

To confirm this estimate, we computed network activity in individual metabolic images from a separate longitudinal cohort of 21 premanifest HD mutation carriers designated HD3 (age: 40.3±6.8 years, range 29-57 years; CAG repeat length: 42.9±2.3, range 39-47; predicted years-to-onset: 11.7±6.5, range 1-25 years) who were scanned twice over a span of 2.3±0.3 years. Like the HD1 longitudinal cohort, this group exhibited a significant linear relationship (FIG. 8B) between the observed increases in pattern expression and “disease time”. Indeed, the rate of network progression estimated for this validation sample (0.19/year (p<0.0001; 95% confidence interval (CI)=[0.11, 0.26], IGM) was nearly identical to that determined for the initial cohort.

Given the stability of these estimates of the preclinical network progression rate, it becomes possible to use this measure as a progression biomarker in placebo-controlled clinical trials of potential disease-modifying agents. Indeed, the longitudinal data acquired in subjects 10 years or less from predicted symptom onset suggest that a 20% difference in progression rate may be detectable with a total sample size as small as 80 gene carriers.

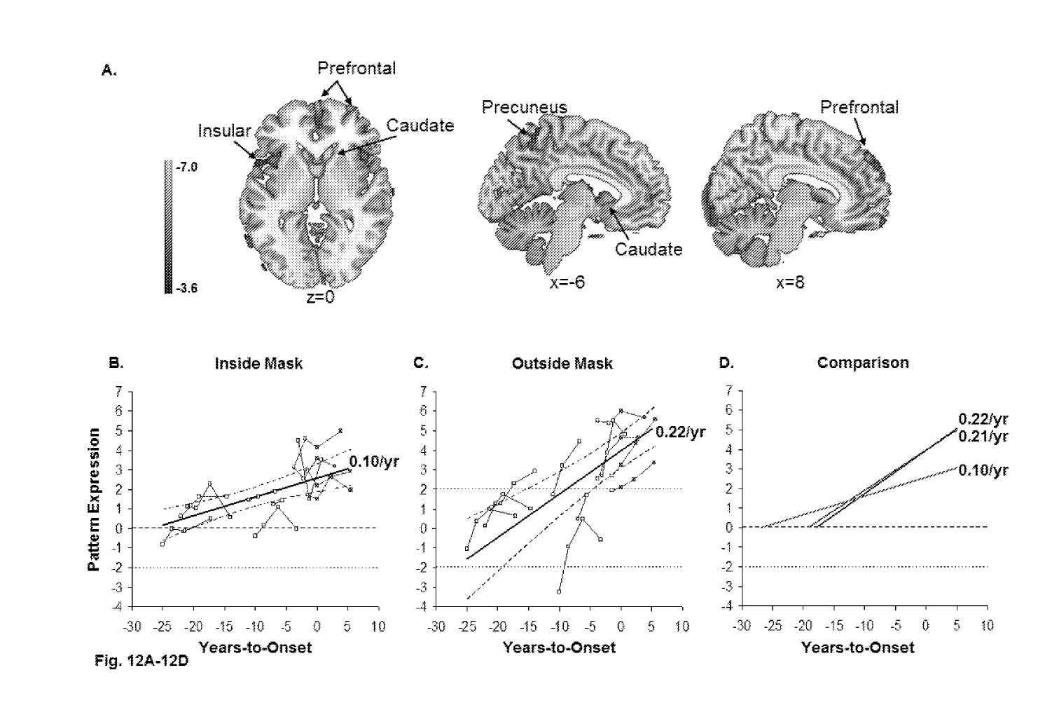

Because HD progression is associated with widespread loss of tissue volume (H. D. Rosas et al. (2008)), we considered the possibility that the measured rate of metabolic progression reflected the concurrent development of localized atrophy in network regions as opposed to systems-level alterations in brain function. To address this issue, we quantified network progression both inside and outside a prespecified volume. A volume-loss mask was defined with voxel-based morphometric (VBM) data acquired from MRI scans made while the subjects were also undergoing metabolic imaging (see Methods). It was found that the mask regions, namely, the ones that lost significant volume over time (FIG. 12A), corresponded closely to the regions previously reported in structural-MRI studies of premanifest gene carriers (H. D. Rosas et al. (2008);, J. S. Paulsen et al. (2002)). Of these atrophic regions (Table 5), the striatum, cerebellum, and prefrontal cortex featured prominently as areas with declining metabolic activity (FIG. 6A) within the HD-progression pattern.

TABLE 5
Brain regions with significant reductions in tissue volume over time

| | | Coordinates^{a} | | |
|---|---|---|---|---|
| Brain region^{b} | | x | y | z |
| Caudate | | 10 | 20 | 0 |
| Prefrontal, dorsolateral (BA 9) | left | −49 | 11 | 31 |
| | right | 45 | 20 | 1 |
| anterior (BA 10) | left | −34 | 56 | 21 |
| | right | 22 | 53 | 41 |

| | | | |
|---|---|---|---|
| Temporal lobe (BA 38) | −64 | −57 | 4 |
| Insula | −39 | 15 | 6 |
| Lateral occipital (BA 19) | −50 | −86 | 0 |
| Parahippocampal gyms | 24 | 5 | −20 |
| Primary somatosensory region (BA 3) | −63 | −20 | −50 |
| Precuneus (BA 7) | 5 | −76 | 36 |

^{a}Montreal Neurological Institute (MNI) standard space (15)
^{b}p < 0.05, false discovery rate (FDR)-corrected (see Methods)
^{c}According to Atlas of Schmahmann (16)
BA = Brodmann Area

That said, the regions with ongoing volume loss did not overlap with network nodes with increasing activity (FIG. 6A), such as the cerebellum, pons, and oribtofrontal cortex. Moreover, significant volume loss was present in several regions not included in the network, such as the primary somatosensory cortex and the precuneus. We also found that network activity both inside (FIG. 12B) and outside (FIG. 12C) the volume-loss mask varied directly with disease time (p<0.0001, IGM). Importantly, however, pattern expression increased twice as fast (0.22 vs. 0.10/year; p<0.004, FIG. 12D) in the part of the network outside the mask (i.e., without major volume loss) as it did inside the atrophic mask. Indeed, the rate of increase in whole-brain pattern activity was nearly identical to that measured in the non-atrophic subspace (0.21 vs. 0.22/year; p=0.90, FIG. 12D). Thus, measurements of network progression across the entire brain volume are not likely to be driven by ongoing regional tissue loss. The data also suggest that formal MRI-based segmentation algorithms for atrophy correction are not necessary as an adjunct to metabolic imaging in determining the network progression rate.

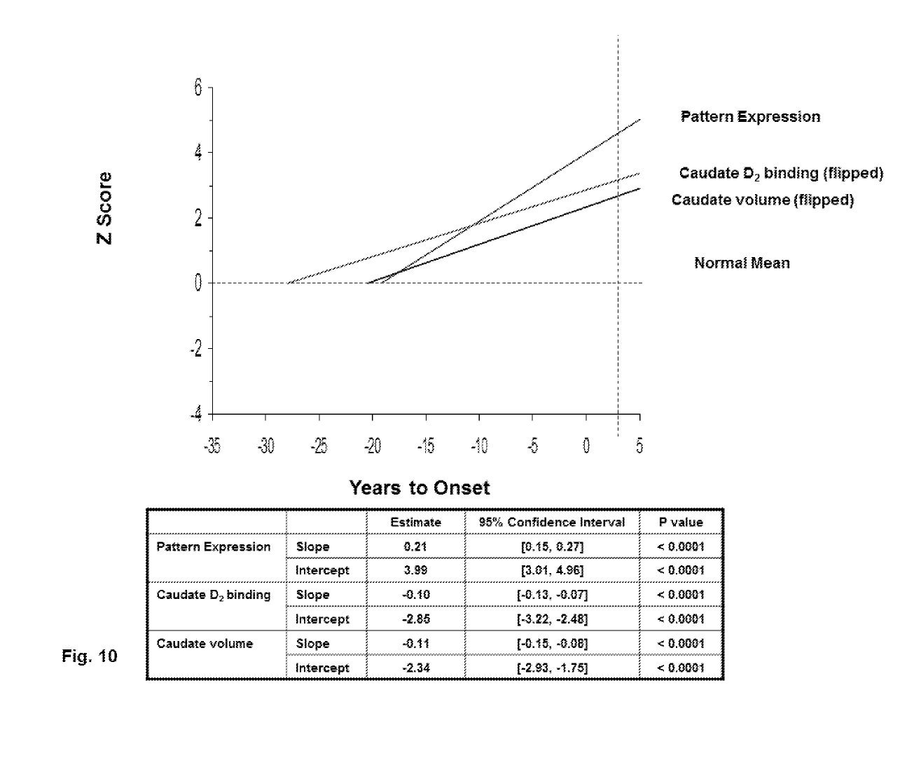

Striatal D₂ Receptor Binding and Tissue Volume. Mean caudate and putamen D₂ table 4 the healthy control groups are presented in Table 6. At baseline, both caudate and putamen D₂-binding values were lower than normal (p<0.005, Student's t-tests), reduced by 35.7% and 33.8%, respectively, of the normal mean. In both regions (FIG. 10A), D₂ receptor binding exhibited a significant linear decline with disease progression (caudate: p<0.0001; putamen: p<0.002, IGM). The rate of decline differed for the two regions (interaction effect: p<0.002), with a faster rate of decline in the caudate (−2.1% of the normal mean per year, 95% CI=[−2.7%, −1.5%]) than in the putamen (−1.8%/year, 95% CI=[−2.9%, −0.8%]). At all time points, caudate and putamen D₂ binding was lower for the HD1 phenoconverters than for the non-phenoconverters. Striatal values for the prospectively imaged symptomatic HD2 subjects were similar to those measured in the four HD1 phenoconverters at seven years.

Mean MRI measurements of caudate and putamen tissue volume at each time point are also presented in Table 6. At baseline, caudate volume was lower in the premanifest HD1 subjects compared to control values (p<0.02, Student's t-test), with a mean reduction of 21.5% below the normal mean. Baseline putamen volume was reduced by 12.4%, which did not differ significantly from normal (p=0.13). Both regions (FIG. 10B) exhibited a significant linear decline in tissue volume (caudate: −2.3% of the normal mean/year, 95% CI=[−2.9%, −1.6%]; putamen: −1.7%/year, 95% CI=[−2.3%, −1.2%]; p<0.0001, IGM) with similar rates of progression (interaction effect: p=0.27). As with caudate and putamen D₂ binding, mean volumes for both striatal regions were lower at all time points for the phenoconverters, and values for the five symptomatic subjects in HD2 were similar to those measured in the four HD1 phenoconverters at seven years. Thus, in keeping with prior studies, we found that striatal D₂ receptor binding declined progressively over time (A. Antonini et al. (1996); A. Antonini et al. (1998)) as did MRI measurements of striatal volume (E. H. Aylward (1998)). Moreover, progression rates were similar for measurements of striatal D₂ receptor binding and tissue volume (caudate: −2.1% vs. −2.3%/year; putamen: −1.8% vs. −1.7%/year).

Natural History of HD in the Preclinical Period. The acquisition of longitudinal multimodal imaging data from premanifest HD carriers enabled a direct comparison of the rate of progression determined for the different measure. Following standardization of each of the imaging descriptors by healthy control values (see Methods), we compared the rate of HD progression estimated from the network activity measurements with corresponding estimates based on concurrent measurements of caudate D₂ binding and tissue volume (FIG. 10). This analysis revealed that the rates of progression estimated from the network approach were significantly greater than the rates derived from the other two, single-region methods (interaction effect: p<0.0001, IGM). The rates of increase in pattern expression measured for the whole brain (0.21/yr) and for the subspace outside the atrophic mask (0.22/yr) were found to be greater than the corresponding rates of change in caudate D₂ binding (−0.10/yr; interaction effect: p<0.0001, IGM) and tissue volume (−0.11/yr; interaction effect: p<0.0005). Of note, estimates of the progression rate based upon striatal D₂ binding and tissue volume measurements were similar whether obtained for the caudate (−0.10 vs. −0.11/yr; p=0.62) or for the putamen (−0.10 vs. −0.09/yr; p=0.46). Interestingly, the two region-level estimates of the progression rate were similar (0.10/yr; p=0.22) to that measured in the part of network with major volume loss (i.e., inside the atrophic mask).

The model also provided reliable estimates for when the various imaging descriptors would begin to deviate from normal, and predicted the likely values of those descriptors during phenoconversion. Thus, the analysis suggested that the observed decline in caudate D₂ binding began approximately 28 years prior to clinical onset (i.e., when years-to-onset=0) and reached an abnormal level (defined as 2 SD below the normal mean, corresponding to a decline to 59% of normal) approximately nine years before phenoconversion. The linear model predicted further 18% decline (to 41% of normal, or 2.9 SD below the normal mean) by the time of diagnosis (intercept: p<0.0001, IGM). Similarly, it was estimated that the decline in caudate volume would have begun approximately 21 years before phenoconversion, reaching abnormal levels (2 SD below the normal mean, corresponding to 61% of normal) approximately three years before phenoconversion. A further decline of 7% (to 54% of normal, or 2.3 SD below the normal mean) was predicted by the time of diagnosis (intercept: p<0.0001, IGM). Interestingly, the data suggest that the decline in caudate D₂ binding began approximately eight years before the start of measureable volume loss in this region, and that the former measure reached abnormally low levels approximately six years earlier than the caudate volume did. Thus, while [¹¹C]-raclopride PET and volumetric MRI provided similar estimates of the rate of decline in the striatal signal, it is likely that D₂ neuroreceptor binding was lost before cell death and the development of atrophy in this brain region (J. H. Cha (2007)).

By contrast, the increase in metabolic network activity was estimated to begin approximately 19 years before clinical onset, coinciding with the start of caudate volume loss. Nonetheless, the network measure was predicted to cross the threshold for abnormal expression (2 SD above the normal mean) approximately 10 years before clinical diagnosis, i.e., seven years before caudate volume loss and at roughly the same time that caudate D₂ receptor binding reached abnormal levels. These estimates are consistent with the finding that the rate of increase in network activity was twice that for the decline in the two region-based imaging measures. The model also predicted that network activity should increase to approximately 4 SD above the normal mean by the onset of clinical HD symptoms (intercept: p<0.0001, IGM). Thus, the network abnormality associated with phenoconversion is of comparatively greater magnitude than the corresponding measures of caudate D₂-receptor binding and tissue volume. Moreover, although loss of striatal D₂ receptor binding may be the earliest observable imaging change in the preclinical period, pattern expression is likely to be more sensitive as a progression biomarker in the decade prior to phenoconversion.

The presence of abnormally elevated network activity at baseline (i.e., subject scores >2.0 at time point 1) was associated with a high likelihood of subsequent phenoconversion. Indeed, each of the four premanifest gene carriers who were ultimately diagnosed with clinical HD (FIG. 7A) had initial pattern expression above this threshold, with an average value of 3.4. Of the eight premanifest HD1 subjects who did not phenoconvert during the follow-up period (FIG. 7A), seven had normal network activity at baseline, with an average value of −0.1. Network activity in these subjects increased, but at comparatively lower levels, during the subsequent time period. One premanifest subject with initially elevated pattern expression (baseline value of 3.9) did not reach clinical diagnosis at the time of final assessment at 3.7 years. Nonetheless, the UHDRS ratings of this subject fluctuated markedly from session to session, a clinical finding consistent with impending phenoconversion. Thus, the individual data suggest the presence of a critical threshold of pattern expression at 2.0 (i.e., 2 SD above the normal mean) above which gene carriers have a substantially higher risk of developing clinical manifestations of HD in the ensuring decade.

In summary, the data demonstrate that subject expression of the HD progression pattern is a sensitive quantitative imaging descriptor of advancing disease in premanifest HD mutation carriers. The progressive increases in network activity observed in preclinical disease can be viewed as an ensemble of stereotyped disease-related regional changes that evolve in the decade before phenoconversion, and which develop further during the period of symptom onset.

### Experimental Results III

Parkinson's disease (PD) and many other disorders, the placebo effect may be one of the most potent but most unstudied clinical phenomena. In any phase 2 clinical trials happening in the United States, it is strictly demanded that any observed real treatment effect (i.e. the actual treatment being tested) should be compared with placebo treatment controls (or sham treatment). While this principal remains unquestioned for most of drug trials, some ethical concerns have been raised as to patients' rights to be offered the best available treatment option (Katsnelson, 2011). It has been proposed that the “real” treatment's effect should be compared to the best available treatment option instead of sham treatments, especially in regard to interventions such as neurosurgery, which can involve burr-holes and implants.

Another drawback of traditional placebo-control study includes the potential risk of burying an effective treatment method. For example in PD, there is no objective biomarker. The gold-standard of clinical outcome still remains to be the physician-evaluated Unified Parkinson's Disease Rating Scale (UPDRS). The situation is even worse for, e.g., Huntington's disease or dystonia. The available subjective rating scales can inflate the variances of the collected data, which makes it difficult to find any statistically significant effect over placebo effect, especially when compared to a potent placebo control such as sham surgery (Goetz et al., 2008). This would be less of a problem if the real treatment effect can be explained by simply additive placebo effect and real treatment effect, but such has not been directly tested. Thus useful treatments, which actually offer benefit over non-treatment, can be abandoned due to the complication of placebo effects.

Polls have suggested the majority of researchers approve placebo-controlled studies (97% supporting sham surgery in one study (Kim et al., 2005)). And not including a placebo control may increase false positives of clinical trials. Therefore, it is preferable to understand the nature of a placebo effect prior to making a decision on whether to include placebo controls or not in clinical trials. In this vein, it has been previously demonstrated that giving placebo increases dopamine release in the striatum in patients with PD (de la Fuente-Fernandez et al., 2001). An analogous phenomenon was also observed in the ventral striatum of test subjects when a sham version of transcranial magnetic stimulation was administered (Strafella et al., 2006). These studies suggest the pivotal role of synaptic dopamine in a short-term placebo effect in PD treatments. No brain imaging studies have explored the long-term effects of sham surgery, which is probably the most ethically controversial topic related with placebo-control (Katsnelson, 2011).

Here, brain metabolic networks were investigated in PD patients who were enrolled in placebo-controlled clinical surgical trials (LeWitt et al., 2011). A placebo effect-related metabolic pattern (PlcRP) was discovered using a supervised multivariate approach (Habeck et al., 2005) on [¹⁸F]-fluorodeoxyglucose (FDG) PET scans which were acquired from subjects before a PD surgery, 6 months after the surgery, and 12 months after the surgery. Other scanning methods, such as fMRI, and other tracers could be used. The pattern expression was later estimated in each individual on a prospective scan basis.

In addition to comparing these measures in the current patient cohort who underwent either real or sham surgery (LeWitt et al., 2011), the network changes that occurred with placebo drug treatment targeting cognitive deficits was evaluated (Mattis et al., 2011), as was natural progression of the disease (Huang et al., 2007) and in response to conventional anti-parkinsonian treatment (Asanuma et al., 2006, Hirano et al., 2008).

### Materials and Methods

Subjects: 23 sham-surgery treated PD patients were studied (17 men and 6 women, age 60.4±1.6 years [mean±SE]) with off-state Unified Parkinson's Disease Rating Scale (UPDRS) motor ratings 37.4±1.8) and 21 PD patients who received real AAV-GAD treatment (16 men and 5 women, age 62.1±1.5 years, off-state UPDRS motor ratings 35.0±1.5) previously reported (LeWitt et al., 2011). Patients were recruited in 6 different sites in the USA. Patients who were initially excluded from the original study were included in the present analysis since their blind was kept for at least for 6 months. All patients were informed that they had a 50% chance of receiving the “real” therapy. All patients and researchers who were involved in PET scans and UPDRS exam were kept blinded at least for 6 months. Six patients in the placebo group (n=23) and 11 patients in the treatment group (n=21) were kept blinded at 12-month follow-up while the rest were unblinded. Written consent was obtained from all patients after detailed explanation of the procedures.

Patients who received placebo surgery were divided into two groups: improved (n=16) and non-improved (n=7), based on changes in UPDRS motor ratings at 6 months after the sham surgery (FIG. 1). Eight patients were selected from the sixteen improved patients and used to derive an FDG spatial covariance pattern that is specific to placebo-induced improvement. The remaining 8 improved and 7 non-improved patients formed the testing group (n=15), and were used to validate the derived pattern.

To identify how the PlcRP expression is affected in other types of placebo treatment study (Mattis et al., 2011), disease progression (Huang et al., 2007) and anti-parkinsonian treatment (Asanuma et al., 2006, Hirano et al., 2008), FDG PET data available from previous studies were also revisited. Patient demographics are reported elsewhere (Asanuma et al., 2006, Hirano et al., 2008, Huang et al., 2007, Mattis et al., 2011).

The brain network-prediction of changes in clinical ratings were performed in re-grouped patients including the patients who received the real AAV-GAD gene therapy. All patients were re-grouped such that GAD group was consist of 16 patients who received successful AAV-GAD treatment and non-GAD group was consist of 23 patients who received sham treatment and 5 patients who received failed real AAV-GAD treatment.

Metabolic imaging: The patients were PET scanned three times with 6 months apart in between: baseline (before surgery), 6-months after surgery, and 12-months after surgery. One patient was not scanned at 12-months. Before each PET session, the patients fasted overnight; antiparkinsonian medications were withheld for at least 12 h before imaging. FDG PET was performed in three dimensional (3D) mode using the GE Advance tomograph (General Electric Medical Systems, Milwaukee, Wis.) at North Shore University Hospital; see Ma and Eidelberg, 2007. The studies were performed with the subjects' eyes open in a dimly lit room and with minimal auditory stimulation.

Scan preprocessing was performed as described elsewhere (Mure et al., 2011). Individual images were warped into MNI standard space using a standard PET template, and smoothed with an isotropic Gaussian kernel (10 mm) in all directions to improve the signal-to-noise ratio.

Network analysis: To identify a specific functional brain network associated with placebo-induced improvement on UPDRS motor ratings, a novel within-subject network modeling strategy was employed. This computational model, termed Ordinal Trends/Canonical Variates Analysis (OrT/CVA, Habeck et al., 2005) is based on supervised principal component analysis and is designed to identify specific spatial covariance patterns in imaging data for which individual measures of subject expression consistently increase or decrease across experimental conditions (e.g., Carbon et al., 2010). OrT/CVA differs from voxel-wise univariate analysis in that it requires that pattern expression values exhibit an “ordinal trend”: the property of consistent change across conditions at the individual subject level. That is, network activity is required to increase (or decrease) monotonically in all or most of the subjects. As in group-wise spatial covariance analysis (e.g., Habeck, 2010, Spetsieris and Eidelberg, 2011), large-scale networks are described in terms of the voxel loadings (“region weights”) on each of the relevant principal component (PC) topographies. Likewise, the expression of a given pattern in each scan is quantified by a specific network activity value (“subject score”), the PC scalar multiplier for the subject in each time. The significance of networks resulting from OrT/CVA is assessed using non-parametric tests. In pattern derivation datasets, permutation tests of the relevant subject scores are used to confirm that the observed monotonic changes in pattern expression across conditions did not occur by chance (p<0.05). The reliability of the voxel loadings comprising the network topography itself is assessed using bootstrap resampling procedures (p<0.05) (Efron and Tibshirani, 1993).

In the current study, a significant placebo-related metabolic pattern (PlcRP) was sought among the linearly independent spatial covariance patterns (i.e., the orthogonal PCs) resulting from OrT/CVA of the scans acquired at baseline (before surgery) and 6 months-after surgery. The following model selection criteria were applied to the individual patterns: (1) the analysis was limited to the first 6 PCs, which typically account for at least 75% of the subject×region variance (Habeck and Stern, 2007); (2) subject scores for these PCs were entered singly and in all possible combinations into a series of logistic regression models, with time (before and 6 months-after) as the dependent variable and the subject scores for each set of PCs as the independent variables for each model. The best model was considered to be that with the smallest Akaike information criterion (AIC) value. The selected PC(s) in this model were then used in linear combination to yield the spatial covariance pattern that was most closely related to the difference across time (before vs. 6 months-after).

To minimize confounds stemming from concurrent effects of disease progression of 6 months, the search of placebo-related network topographies was restricted to the portion of the subject×voxel space that was independent of (i.e., orthogonal to) a pre-specified subspace known empirically to be associated with PD. This was accomplished by orthogonalization to the PDRP, a previously described SSM/PCA topography identified from the mixture of 33 patients with PD and 33 normal controls (Ma et al., 2007). Before orthogonalization to PDRP, it was verified that a consistent and statistically significant increase of PDRP at 6 month was evident in the current dataset. The spatial covariance pattern for real AAV-GAD treatment effect (GADP) has also been identified elsewhere, which has shown significant correlation between GADP expression and clinical benefits in the treated patients.

Statistical data analysis: To validate if the derived pattern, i.e., PlcRP, was able to identify the differences between the patients who showed improvement 6 months after the sham surgery (n=8, not included in the derivation group) and those who did not (n=7), an independent t-test was performed between improved and non-improved patients group. Further, to see if the changes in PlcRP are correlated with clinical improvement, Pearson's correlation was tested between changes in PlcRP and changes in UPDRS motor ratings in each group (derivation group, improved and non-improved). In addition, to determine if the baseline subject scores of PlcRP or UPDRS motor rating predict the placebo-response, baseline measures (PlcRP and UPDRS) were tested for Pearson's correlation with their subsequent changes after 6 months. Finally, in order to see the effect of unblinding (6 patients kept blinded at 12 month and 16 patients were unblinded at 12 month), 2×3 repeated measures ANOVA was performed on UPDRS motor ratings and PlcRP scores (group×time).

The relationship between GADP and PlcRP in respect to the changes in clinical ratings (UPDRS motor scores) are analyzed with general linear model (GLM) (McClullagh and Nelder, 1989). The following linear models were evaluated for the two groups (GAD group and non-GAD group) separately:

UPDRS=B*subjects[2 . . . n]+c

UPDRS=B*subjects[2 . . . ]+b1*GADP+c

UPDRS=B*subjects[2 . . . n]+b1*PlcRP+c

UPDRS=B*subjects[2 . . . n]+b1*GADP+b2*PlcRP+c

The best model was considered to be that with the smallest AIC value.

To examine if the PlcRP reflect multi-dimensional spectrum of PD symptoms, e.g., the cognitive deficits, independent t-test was performed on PlcRP scores between placebo-responder (n=7) vs. non-responder (n=5). In this study (Mattis et al., 2011), twelve patients with PD were treated with placebo for two months. The subjects were told that the objective of the study was to examine the effect of Donepezil on cognitive deficits in PD. Patients were told that they have 50% chances to be treated with real drug. Patients were divided to responder (n=7) and non-responder (n=5) based on meaningful cognitive improvement. For details, see Mattis et al. (2011).

To examine the effect of disease progression and anti-parkinsonian treatment on PlcRP expression, paired t-test was performed. In the disease progression study (Huang et al., 2007), 15 patients were scanned with FDG PET at baseline and −2 year after. In the treatment study (Asanuma et al., 2006, Hirano et al., 2008), 11 patients were scanned with FDG PET on and off levodopa infusion, and 13 patients were scanned with FDG PET on and off STN-DBS. For details, see Huang et al. (2007), Asanuma et al. (2006) and Hirano et al. (2008).

### Results

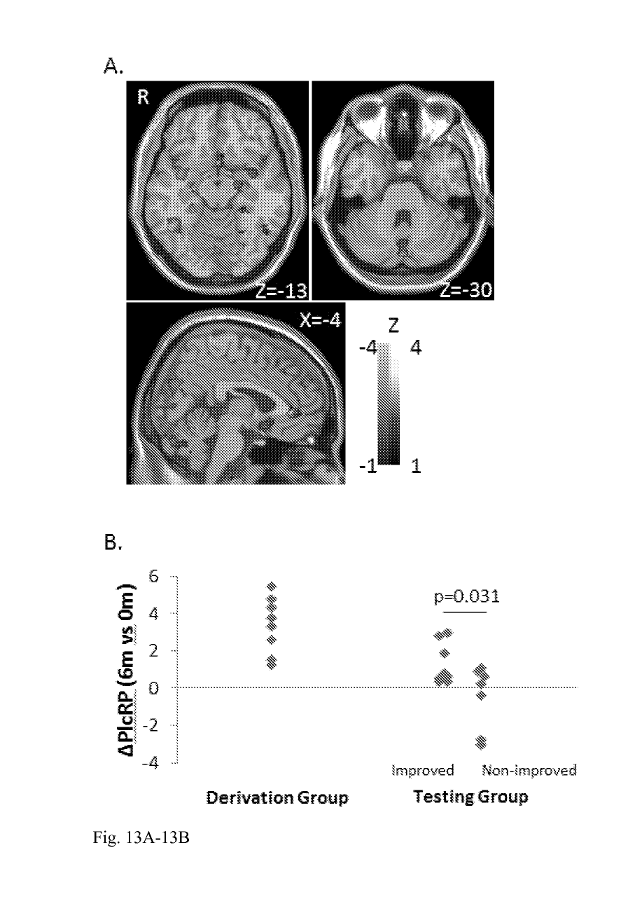

Placebo-related spatial covariance pattern: The OrT/CVA identified that the best model fit was achieved (smallest AIC) with the linear combination of PC3 and PC4. Within the derivation group (n=8), no exception was reported in the ordinal trend, i.e., all patients subject score was increased at 6 month compared to the baseline (before surgery). Several regions were identified to have increased FDG uptake including subgenual anterior cingulate cortex, cerebellar vermis, inferior temporal cortex, hippocampus and amygdala (FIG. 13A) (Table 6). Regions with decreased FDG uptake at 6 months included inferior temporal, parahippocampal gyms and cuneus (Table 6). The voxel weights in these regions were stable by bootstrap estimation (p<0.05). The permutation of subject images across time revealed that the derived pattern did not occur by chance (p<0.001).

TABLE 6
Regions and the peak coordinates that
are identified in PlcRP by OrT/CVA.

| | | | | | |
|---|---|---|---|---|---|
| Region | BA | x | y | z | Z_{intensity} |

Hyperactivity

| | | | | | |
|---|---|---|---|---|---|
| Anterior Cingulate | 32/24 | 2 | 32 | −2 | 3.95 |
| Cortex | Bilateral | | | | |
| Subcallosal gyrus | 25 Right | 2 | 10 | −16 | 2.83 |
| Inferior Temporal | 37 Left | −44 | −56 | −8 | 4.44 |
| (fusiform/ | | | | | |
| parahippocampal) | | | | | |
| Hippocampus | 19/37 Right | 30 | −46 | −22 | 3.03 |
| | Left | −22 | −14 | −12 | 3.43 |
| | Right | 20 | −12 | −12 | 2.40 |
| Amygdala (extends | Right | 32 | −2 | −16 | 2.08 |
| to inferior temporal) | | | | | |
| Cerebellum (Vermis) | Bilateral | −2 | −82 | −28 | 3.37 |

Hypoactivity

| | | | | | |
|---|---|---|---|---|---|
| Inferior Temporal | 37/20 Right | 60 | −54 | −20 | −2.66 |
| Occipital/Temporal | 19/39 Left | −52 | −76 | 8 | −2.67 |
| Cuneus | Left | −6 | −82 | 30 | −2.63 |
| Parahippocampal | Left | −24 | −40 | −8 | −3.85 |

*Voxel loadings of the reported regions are reliable by bootstrapping (p < 0.05).

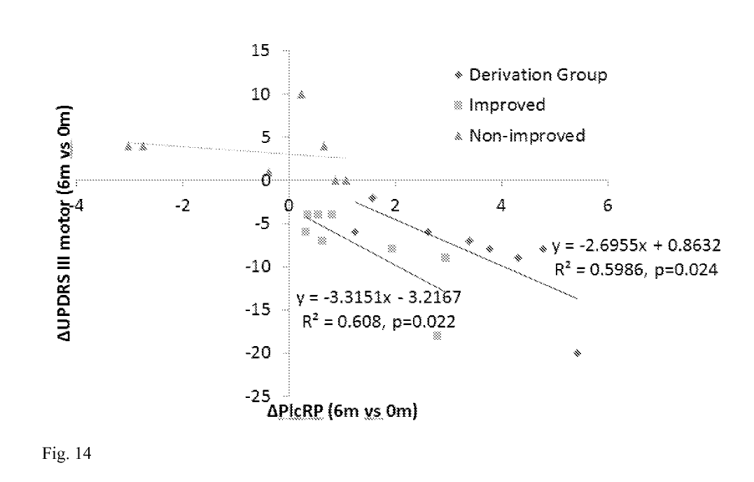

Validation of PlcRP and correlation with UPDRS: All patients' PlcRP subject scores were increased in the derivation group (n=8; FIG. 3B). In the testing group, all improved patients' PlcRP subject scores were increased (n=8), while only 4 out of 7 non-improved patients' PlcRP subject scores were increased (FIG. 13B). Difference between improved and non-improved patients' PlcRP subject scores were significant within the testing group (413)=2.413, p=0.031). Significant negative correlation between changes in UPDRS motor ratings and PlcRP scores was observed within the derivation group (r=−0.774, p=0.024) and improved patients in testing group (r=−0.780, p=0.022) (FIG. 14). No significant correlation was observed in the patients whose UPDRS motor rating was not changed or worsened (r=−0.211, p=0.650).

Relationship of PlcRP with real treatment-related network and UPDRS scores: When the groups of subjects are reorganized according to the unblinding at 12 months after the surgery, the 2×3 repeated measures ANOVA (group×time) revealed significant main effect of time (0 m, 6 m, 12 m) in UPDRS motor ratings (f(2,40)=4.367, p=0.019) and PlcRP scores (f(2,40)=7.246, p=0.002). However no significant interaction effect (blinding vs time) was observed with either UPDRS motor ratings (f(2,40)=0.473, p=0.627) or PlcRP scores (f(2,40)=1.039, p=0.363).

In the group of patients who are assigned to receive the real treatment, 15 out of 21 patients showed increase of PlcRP scores at 6 month which is similar to the patients who received placebo (cf. 16 out of 23 patients). However changes PlcRP was not correlated with changes in UPDRS motor ratings (r=−0.149, p=0.519, Figure S2).

In the GLM analysis of UPDRS prediction model, the changes in PlcRP expression significantly predicted the changes in UPDRS motor ratings (p=0.0027) while the changes in GADP expression did not (p=0.56) in the non-GAD group (Table 7).

TABLE 7
Prediction of clinical benefits (UPDRS-III) from network
scores at 0 m, 6 m and 12 m in non-GAD group (n = 28)

| | Model fit | | | | Predictor variables | | |
|---|---|---|---|---|---|---|---|
| predictor | Dfe | adj. r2 | p | AIC | b | t | p |
| GADP | 54 | 0.007 | 0.56 | 556.5 | −0.44 | −0.59 | 0.56 |
| PlcRP | 54 | 0.155 | 0.003 | 543.0* | −1.69 | −3.15 | 0.003 |
| GADP | 53 | 0.162 | 0.005 | 544.3 | 0.50 | 0.66 | 0.51 |
| PlcRP | | | | | −1.84 | −3.14 | 0.003 |

Observed response: UPDRS-III
*The model with PlcRP alone showed the lowest AIC-value.

Conversely in the GAD group, only the changes in GADP expression significantly predicted the changes in UPDRS motor ratings (p<0.001) while the changes in PlcRP expression did not (p=0.090) (Table 8). There was no additive effect on the prediction model when the two pattern expressions were entered in the GLM, i.e., AIC-value was the smallest when the PlcRP alone predicted UPDRS in the non-GAD group while the GADP alone predicted the UPDRS changes in the GAD group (Tables 7, 8).

TABLE 8
Prediction of clinical benefits (UPDRS-III) from network
scores at 0 m, 6 m and 12 m in GAD group (n = 16)

| | Model fit | | | | Predictor variables | | |
|---|---|---|---|---|---|---|---|
| predictor | dfe | adj. r2 | p | AIC | b | t | p |
| GADP | 30 | 0.469 | <0.001 | 293.8* | −2.07 | −5.14 | <0.001 |
| PlcRP | 30 | 0.093 | 0.090 | 319.0 | −1.57 | −1.75 | 0.090 |
| GADP | 29 | 0.472 | <0.001 | 295.5 | −2.17 | −4.56 | <0.001 |
| PlcRP | | | | | 0.33 | 0.41 | 0.685 |

Observed response: UPDRS-III
*The model with GADP alone showed the lowest AIC-value.

### Discussion

The OrT/CVA successfully derived a spatial metabolic pattern that is related with placebo-induced clinical improvement measured by UPDRS motor ratings. All patients whose UPDRS motor ratings were improved after 6 months showed increased PlcRP scores (FIG. 13B). However, three out of seven patients whose UPDRS motor score was increased also showed increased PlcRP expression. Thus, the sensitivity was 100% in the present small sample size of the testing group (n=15). The most intriguing finding was that the increased PlcRP scores were correlated with clinical improvement (FIG. 14).

In the real treatment group (n=21), similar percentage of patients showed increase of PlcRP scores as in the placebo group (placebo: 69.6%, real: 71.4%). However, unlike the placebo group, this change was not correlated with UPDRS motor ratings (Table 8), possibly due to its less significant effect compared the real treatment. In other words, sub-group of patients in the real treatment group also expressed some degree of PlcRP, but the effect of real treatment on UPDRS motor ratings was far greater than the effect of expectation of benefit which is reflected by PlcRP, thus it abolished the correlation between changes PlcRP and changes in UPDRS motor ratings. This result strengthens the conclusion that the previously reported benefit of GAD treatment is distinct from placebo effect (LeWitt et al., 2011).

Since PlcRP scores were not changed by disease progression, it is understood the changes in PlcRP expression and its correlation with clinical benefits do not reflect natural compensatory mechanisms that evolve as the disease progresses. In addition, conventional anti-parkinsonian treatment (i.e., levodopa and STN DBS) did not affect PlcRP expression, thus its long-term clinical benefit may be achieved via non-dopaminergic pathway which is not directly involved with cortico-basal ganglia output circuitry (cf., short-term placebo effect has been shown to be associated with striatal dopamine release; de la Fuente-Fernandez et al., 2001, Lidstone et al., 2010, Strafella et al., 2006). Instead, PlcRP topography suggests the significant contribution of limbic-cerebellar network (FIG. 13A; Table 7) which may be involved with reward/reinforcement circuitry.

Previous studies with placebo effects on other spectrum reported some overlapping but inconsistent regional involvement, e.g., increased activity in the subgenual ACC and hippocampus/parahippocampus in depression (Mayberg et al., 2002) and increased/decreased activity in the rostral ACC in pain (Petrovic et al., 2002, Wager et al., 2004). While no meta-analysis have been performed in different spectrum of placebo effects, no evidence of common and consistent contribution of specific regions in placebo effects have been documented.

The methodological implication of this study may suggest overall revision of requirement of placebo control groups in the clinical trials. When the real treatment effect and placebo effect can be identified in separate brain metabolic patterns which separately correlate with clinical benefits, it is not necessary to have equivalent number of patients to show the statistical difference between the two groups. For example, 1:3 ratio of patients enrolled in the placebo control group compared to the patients enrolled in real treatment group could be employed. The difference in underlying mechanisms of clinical benefits between real vs. sham treatment then can be explained by neuroimaging measures such as PlcRP vs GADP (Table 7/8).

### REFERENCES

Antonini, A., Moeller, J. R., Nakamura, T., Spetsieris, P., Dhawan, V., Eidelberg, D., 1998. The metabolic anatomy of tremor in Parkinson's disease. Neurology 51, 803-810.
Asanuma, K., Tang, C., Ma, Y., Dhawan, V., Mattis, P., Edwards, C., Kaplitt, M. G., Feigin, A., Eidelberg, D., 2006. Network modulation in the treatment of Parkinson's disease Brain 129, 2667-2678.
Bair E, H. T., Paul D, Tibshirani R, 2006. Prediction by supervised principal components. J Am Stat Assoc 101, 119-137.
Baron, J. C., Levasseur, M., Mazoyer, B., Legault-Demare, F., Mauguiere, F., Pappata, S., Jedynak, P., Derome, P., Cambier, J., Tran-Dinh, S., et al., 1992. Thalamocortical diaschisis: positron emission tomography in humans. J Neurol Neurosurg Psychiatry 55, 935-942.
Benamer, H. T., Oertel, W. H., Patterson, J., Hadley, D. M., Pogarell, O., Hoffken, H., Gerstner, A., Grosset, D. G., 2003. Prospective study of presynaptic dopaminergic imaging in patients with mild parkinsonism and tremor disorders: part 1. Baseline and 3-month observations. Mov Disord 18, 977-984.
Blahak, C., Wohrle, J. C., Capelle, H. H., Bazner, H., Grips, E., Weigel, R., Hennerici, M. G., Krauss, J. K., 2007. Tremor reduction by subthalamic nucleus stimulation and medication in advanced Parkinson's disease. J Neurol 254, 169-178.
Boecker, H., Brooks, D. J., 1998. Functional imaging of tremor. Movement Disorders 13 Suppl 3, 64-72.
Boecker, H., Wills, A. J., Ceballos-Baumann, A., Samuel, M., Thomas, D. G., Marsden, C. D., Brooks, D. J., 1997. Stereotactic thalamotomy in tremor-dominant Parkinson's disease: an H2(15)O PET motor activation study. Annals of Neurology 41, 108-111.
Bostan, A. C., Dum, R. P., Strick, P. L., 2010. The basal ganglia communicate with the cerebellum. Proc Natl Acad Sci USA 107, 8452-8456.
Carbon, M., Argyelan, M., Habeck, C., Ghilardi, M. F., Fitzpatrick, T., Dhawan, V., Pourfar, M., Bressman, S. B., Eidelberg, D., 2010. Increased sensorimotor network activity in DYT1 dystonia: a functional imaging stud. Brain 133, 690-700.
Deiber, M. P., Pollak, P., Passingham, R., Landais, P., Gervason, C., Cinotti, L., Friston, K., Frackowiak, R., Mauguiere, F., Benabid, A. L., 1993. Thalamic stimulation and suppression of parkinsonian tremor. Evidence of a cerebellar deactivation using positron emission tomography. Brain 116 (Pt 1), 267-279.
DeLong, M. R., Wichmann, T., 2007. Circuits and circuit disorders of the basal ganglia. Arch Neurol 64, 20-24.
Deuschl, G., Raethjen, J., Baron, R., Lindemann, M., Wilms, H., Krack, P., 2000. The pathophysiology of parkinsonian tremor: a review. Journal of Neurology 247 Suppl 5, V33-48.
Deuschl, G., Raethjen, J., Lindemann, M., Krack, P., 2001. The pathophysiology of tremor. Muscle Nerve 24, 716-735.
Eckert, T., Van Laere, K., Tang, C., Lewis, D. E., Edwards, C., Santens, P., Eidelberg, D., 2007. Quantification of Parkinson's disease-related network expression with ECD SPECT. Eur J Nucl Med Mol Imaging 34, 496-501.
Eidelberg, D., 2009. Metabolic brain networks in neurodegenerative disorders: a functional imaging approach. Trends Neurosci 32, 548-557.
Eidelberg, D., Moeller, J. R., Dhawan, V., Spetsieris, P., Takikawa, S., Ishikawa, T., Chaly, T., Robeson, W., Margouleff, D., Przedborski, S., 1994. The metabolic topography of parkinsonism. Journal of Cerebral Blood Flow & Metabolism 14, 783-801.
Eidelberg, D., Moeller, J. R., Ishikawa, T., Dhawan, V., Spetsieris, P., Chaly, T., Belakhlef, A., Mandel, F., Przedborski, S., Fahn, S., 1995a. Early differential diagnosis of Parkinson's disease with 18F-fluorodeoxyglucose and positron emission tomography. Neurology 45, 1995-2004.
Eidelberg, D., Moeller, J. R., Ishikawa, T., Dhawan, V., Spetsieris, P., Chaly, T., Robeson, W., Dahl, J. R., Margouleff, D., 1995b. Assessment of disease severity in parkinsonism with fluorine-18-fluorodeoxyglucose and PET. J Nucl Med 36, 378-383.
Eidelberg, D., Moeller, J. R., Kazumata, K., Antonini, A., Sterio, D., Dhawan, V., Spetsieris, P., Alterman, R., Kelly, P. J., Dogali, M., Fazzini, E., Beric, A., 1997. Metabolic correlates of pallidal neuronal activity in Parkinson's disease. Brain 120, 1315-1324.
Fahn S and Elton R, M.o.t.U.D.C., 1987. In: Fahn S, Marsden C D, Calne D B, Goldstein M, eds. Recent Developments in Parkinson's Disease, Vol 2. Florham Park, N.J. Macmillan Health Care Information, 153-163.
Feigin, A., Fukuda, M., Dhawan, V., Przedborski, S., Jackson-Lewis, V., Mentis, M. J., Moeller, J. R., Eidelberg, D., 2001. Metabolic correlates of levodopa response in Parkinson's disease. Neurology 57, 2083-2088.
Fishman, P. S., 2008. Paradoxical aspects of parkinsonian tremor. Movement Disorders 23, 168-173.
Friston, K. J., Penny, W. D., Glaser, D. E., 2005. Conjunction revisited. Neuroimage 25, 661-667.
Fukuda, M., Barnes, A., Simon, E. S., Holmes, A., Dhawan, V., Giladi, N., Fodstad, H., Ma, Y., Eidelberg, D., 2004. Thalamic stimulation for parkinsonian tremor: correlation between regional cerebral blood flow and physiological tremor characteristics. Neuroimage 21, 608-615.
Habeck, C., Krakauer, J. W., Ghez, C., Sackeim, H. A., Eidelberg, D., Stern, Y., Moeller, J. R., 2005. A new approach to spatial covariance modeling of functional brain imaging data: ordinal trend analysis. Neural Computation 17, 1602-1645.
Habeck, C., Stern, Y., 2007. Neural network approaches and their reproducibility in the study of verbal working memory and Alzheimer's disease. Clin Neurosci Res 6, 381-390.
Haslinger, B., Boecker, H., Buchel, C., Vesper, J., Tronnier, V. M., Pfister, R., Alesch, F., Moringlane, J. R., Krauss, J. K., Conrad, B., Schwaiger, M., Ceballos-Baumann, A. O., 2003. Differential modulation of subcortical target and cortex during deep brain stimulation. Neuroimage 18, 517-524.
Herzog, J., Hamel, W., Wenzelburger, R., Potter, M., Pinsker, M. O., Bartussek, J., Morsnowski, A., Steigerwald, F., Deuschl, G., Volkmann, J., 2007. Kinematic analysis of thalamic versus subthalamic neurostimulation in postural and intention tremor. Brain 130, 1608-1625.
Hirano, S., Asanuma, K., Ma, Y., Tang, C., Feigin, A., Dhawan, V., Carbon, M., Eidelberg, D., 2008. Dissociation of metabolic and neurovascular responses to levodopa in the treatment of Parkinson's disease. J Neurosci 28, 4201-4209.
Hoshi, E., Tremblay, L., Feger, J., Carras, P. L., Stick, P. L., 2005. The cerebellum communicates with the basal ganglia. Nature Neuroscience 8, 1491-1493.
Huang, C., Mattis, P., Tang, C., Perrine, K., Carbon, M., Eidelberg, D., 2007a. Metabolic brain networks associated with cognitive function in Parkinson's disease. Neuroimage 34, 714-723.
Huang, C., Tang, C., Feigin, A., Lesser, M., Ma, Y., Pourfar, M., Dhawan, V., Eidelberg, D., 2007b. Changes in network activity with the progression of Parkinson's disease. Brain 130, 1834-1846.
Hughes, A. J., Daniel, S. E., Blankson, S., Lees, A. J., 1993. A clinicopathologic study of 100 cases of Parkinson's disease. Archives of Neurology 50, 140-148.
Isaias, I., Marotta, G., Hirano, S., 2010. Imaging Essential Tremor. Movement Disorders 25, 679-686.
Ishikawa, T., Dhawan, V., Kazumata, K., Chaly, T., Mandel, F., Neumeyer, J., Margouleff, C., Babchyck, B., Zanzi, I., Eidelberg, D., 1996. Comparative nigrostriatal dopaminergic imaging with iodine-123-beta CIT-FP/SPECT and fluorine-18-FDOPA/PET. J Nucl Med 37, 1760-1765.
Kazumata, K., Antonini, A., Dhawan, V., Moeller, J. R., Alterman, R. L., Kelly, P., Sterio, D., Fazzini, E., Beric, A., Eidelberg, D., 1997. Preoperative indicators of clinical outcome following stereotaxic pallidotomy. Neurology 49, 1083-1090.
Lenz, F. A., Kwan, H. C., Martin, R. L., Tasker, R. R., Dostrovsky, J. O., Lenz, Y. E., 1994. Single unit analysis of the human ventral thalamic nuclear group. Tremor-related activity in functionally identified cells. Brain 117 (Pt 3), 531-543.
Lin, T. P., Carbon, M., Tang, C., Mogilner, A. Y., Sterio, D., Beric, A., Dhawan, V., Eidelberg, D., 2008. Metabolic correlates of subthalamic nucleus activity in Parkinson's disease. Brain 131, 1373-1380.
Louis, E. D., Tang, M. X., Cote, L., Alfaro, B., Mejia, H., Marder, K., 1999. Progression of parkinsonian signs in Parkinson disease. Arch Neurol 56, 334-337.
Lozza, C., Baron, J. C., Eidelberg, D., Mentis, M. J., Carbon, M., Marie, R. M., 2004 Executive processes in Parkinson's disease: FDG-PET and network analysis. Hum Brain Mapp 22, 236-245.
Lyons, K. E., Koller, W. C., Wilkinson, S. B., Pahwa, R., 2001. Long term safety and efficacy of unilateral deep brain stimulation of the thalamus for parkinsonian tremor. J Neurol Neurosurg Psychiatry 71, 682-684.
Ma, Y., Eidelberg, D., 2007. Multivariate brain mapping in clinical neuroscience research. Clin Neurosci Res 6, 357-358.
Ma, Y., Huang, C., Dyke, J. P., Pan, H., Alsop, D., Feigin, A., Eidelberg, D., 2010. Parkinson's disease spatial covariance pattern: noninvasive quantification with perfusion MRI J Cereb Blood Flow Metab 30, 505-509.
Ma, Y., Tang, C., Spetsieris, P. G., Dhawan, V., Eidelberg, D., 2007. Abnormal metabolic network activity in Parkinson's disease: test-retest reproducibility. J Cereb Blood Flow Metab 27, 597-605.
Machado, A., Rezai, A. R., Kopell, B. H., Gross, R. E., Sharan, A. D., Benabid, A. L., 2006. Deep brain stimulation for Parkinson's disease: surgical technique and perioperative management. Mov Disord 21 Suppl 14, S247-258.
Martinez-Martin, P., Gil-Nagel, A., Gracia, L. M., Gomez, J. B., Martinez-Sarries, J., Bermejo, F., 1994. Unified Parkinson's Disease Rating Scale characteristics and structure. The Cooperative Multicentric Group. Mov Disord 9, 76-83.
Middleton, F. A., Strick, P. L., 2000. Basal ganglia and cerebellar loops: motor and cognitive circuits. Brain Research—Brain Research Reviews 31, 236-250.
Moeller, J., Habeck, C., 2006. Reciprocal Benefits of Mass-Univariate and Multivariate Modeling in Brain Mapping: Applications to event-related functional MRI, H215O-, and FDG PET. International Journal of Biomedical Imaging 2006, 1-13.
Nambu, A., 2004. A new dynamic model of the cortico-basal ganglia loop. Prog Brain Res 143, 461-466.
Ohye, C., Shibazaki, T., Hirai, T., Wada, H., Kawashima, Y., Hirato, M., Matsumura, M., 1988. A special role of the parvocellular red nucleus in lesion-induced spontaneous tremor in monkeys. Behavioural Brain Research 28, 241-243.
Pechadre, J. C., Larochelle, L., Poirier, L. J., 1976. Parkinsonian akinesia, rigidity and tremor in the monkey. Histopathological and neuropharmacological study. Journal of the Neurological Sciences 28, 147-157.
Perlmutter, J. S., Mink, J. W., Bastian, A. J., Zackowski, K., Hershey, T., Miyawaki, E., Koller, W., Videen, T. O., 2002. Blood flow responses to deep brain stimulation of thalamus. Neurology 58, 1388-1394.
Poirier, L. J., Sourkes, T. L., Bouvier, G., Boucher, R., Carabin, S., 1966. Striatal amines, experimental tremor and the effect of harmaline in the monkey. Brain 89, 37-52.
Pourfar, M., Tang, C., Lin, T., Dhawan, V., Kaplitt, M. G., Eidelberg, D., 2009. Assessing the microlesion effect of subthalamic deep brain stimulation surgery with FDG PET. J Neurosurg 110, 1278-1282.
Rajput, A. H., Rozdilsky, B., Ang, L., 1991. Occurrence of resting tremor in Parkinson's disease. Neurology 41, 1298-1299.
Rehncrona, S., Johnels, B., Widner, H., Tornqvist, A. L., Hariz, M., Sydow, O., 2003. Longterm efficacy of thalamic deep brain stimulation for tremor: double-blind assessments. Mov Disord 18, 163-170.
Rezai, A. R., Lozano, A. M., Crawley, A. P., Joy, M. L., Davis, K. D., Kwan, C. L., Dostrovsky, J. O., Tasker, R. R., Mikulis, D. J., 1999. Thalamic stimulation and functional magnetic resonance imaging: localization of cortical and subcortical activation with implanted electrodes. Technical note. J Neurosurg 90, 583-590.
Schmahmann, J. D., Doyon, J., Toga, A. W., Petrides, M., Evans, A. C. MRI atlas of the human cerebellum. San Diego: Academic Press, 2000.
Singer, J. D., Willett, J. B. Applied longitudinal data analysis: modeling change and event occurrence. Oxford: Oxford University Press, 2003.
Spetsieris, P. G., Ma, Y., Dhawan, V., Eidelberg, D., 2009. Differential diagnosis of parkinsonian syndromes using PCA-based functional imaging features. Neuroimage 45, 1241-1252.
Stochl, J., Boomsma, A., Ruzicka, E., Brozova, H., Blahus, P., 2008. On the structure of motor symptoms of Parkinson's disease. Mov Disord 23, 1307-1312.
Suckling, J., Bullmore, E., 2004. Permutation tests for factorially designed neuroimaging experiments. Hum Brain Mapp 22, 193-205.
Tang, C. C., Poston, K. L., Dhawan, V., Eidelberg, D., 2010. Abnormalities in metabolic network activity precede the onset of motor symptoms in Parkinson's disease. J Neurosci 30, 1049-1056.
Timmermann, L., Florin, E., Reck, C., 2007. Pathological cerebral oscillatory activity in Parkinson's disease: a critical review on methods, data and hypotheses. Expert Rev Med Devices 4, 651-661.
Timmermann, L., Gross, J., Dirks, M., Volkmann, J., Freund, H. J., Schnitzler, A., 2003. The cerebral oscillatory network of parkinsonian resting tremor. Brain 126, 199-212.
Tro\check{s}t, M., Su, S., Su, P., Yen, R. F., Tseng, H. M., Barnes, A., Ma, Y., Eidelberg, D., 2006. Network modulation by the subthalamic nucleus in the treatment of Parkinson's disease. Neuroimage 31, 301-307.
Volkmann, J., Joliot, M., Mogilner, A., Ioannides, A. A., Lado, F., Fazzini, E., Ribary, U., Llinas, R., 1996. Central motor loop oscillations in parkinsonian resting tremor revealed by magnetoencephalography. Neurology 46, 1359-1370.
West, S. G., Aiken, L. S., Krull, J. L., 1996. Experimental personality designs: analyzing categorical by continuous variable interactions. J Pers 64, 1-48.
Wielepp, J. P., Burgunder, J. M., Pohle, T., Ritter, E. P., Kinser, J. A., Krauss, J. K., 2001. Deactivation of thalamocortical activity is responsible for suppression of parkinsonian tremor by thalamic stimulation: a 99mTc-ECD SPECT study. Clin Neurol Neurosurg 103, 228-231.
Zaidel, A., Arkadir, D., Israel, Z., Bergman, H., 2009. Akineto-rigid vs. tremor syndromes in Parkinsonism. Curr Opin Neurol 22, 387-393.
J. S. Paulsen, Exp Neurol 216, 272 (April, 2009).
D. Eidelberg, D. J. Surmeier, J Clin Invest 121, 484 (2011).
M. Esmaeilzadeh, A. Ciarmiello, F. Squitieri, CNS Neurosci Ther, (Jun. 11, 2010).
D. Eidelberg, Trends Neurosci 32, 548 (October, 2009).
C. C. Tang, K. L. Poston, V. Dhawan, D. Eidelberg, J Neurosci 30, 1049 (Jan. 20, 2010).
A. Feigin et al., Brain 130, 2858 (November, 2007).
J. R. Moeller, C. G. Habeck, Int J Biomed Imag 2006, 1 (2006).
C. Habeck et al., Neural Comput 17, 1602 (July, 2005).
E. H. Aylward et al., Neurology 48, 394 (February, 1997).
B. G. Jenkins et al., J Neurochem 95, 553 (October, 2005).
A. Feigin et al., Ann Neurol 59, 53 (January, 2006).
H. D. Rosas et al., Neurology 58, 695 (Mar. 12, 2002).
H. D. Rosas, A. S. Feigin, S. M. Hersch, NeuroRx 1, 263 (April, 2004).
H. D. Rosas et al., Ann N Y Acad Sci 1147, 196 (December, 2008).
J. S. Paulsen et al., Am J Neuroradiol 25, 1715 (November-December, 2004).
A. Antonini et al., Brain 119, 2085 (1996).
A. Antonini, K. L. Leenders, D. Eidelberg, Ann Neurol 43, 253 (February, 1998).
E. H. Aylward, Brain Res Bull 72, 152 (Apr. 30, 2007).
J. H. Cha, Prog Neurobiol 83, 228 (November, 2007).
D. L. Collins, P. Neelin, T. M. Peters, A. C. Evans, J Comput Assist Tomogr 18, 192 (March-April, 1994).
J. D. Schmahmann, J. Doyon, A. W. Toga, M. Petrides, A. C. Evans, MRI atlas of the human cerebellum. (Academic Press, San Diego, 2000).
Mov Disord 11, 136 (March, 1996).
A. Feigin et al., Brain 130, 2858 (November, 2007).
J. C. van Oostrom et al., Neurology 65, 941 (Sep. 27, 2005).
J. C. van Oostrom et al., Eur J Neurol 16, 226 (February, 2009).
Y. Ma, C. Tang, P. G. Spetsieris, V. Dhawan, D. Eidelberg, J Cereb Blood Flow Metab 27, 597 (March, 2007).
C. Huang et al., Brain 130, 1834 (July, 2007).
C. Habeck et al., Neural Comput 17, 1602 (July, 2005).
J. R. Moeller, C. G. Habeck, Int J Biomed Imag 2006, 1 (2006).
C. Habeck, Y. Stern, Cell Biochem Biophys 58, 53 (November, 2010).
B. Efron, R. Tibshirani, An introduction to the bootstrap., (CRC Press, LLC, New York, 1994).
K. P. Burnham, D. R. Anderson, Model Selection and Multimodel Inference: A Practical Information-Theoretic Approach, 2nd ed. (Springer-Verlag, 2002).
S. S. Keller, M. Wilke, U. C. Wieshmann, V. A. Sluming, N. Roberts, Neuroimage 23, 860 (November, 2004).
H. H. Ruocco, L. Bonilha, L. M. Li, I. Lopes-Cendes, F. Cendes, J Neurol Neurosurg Psychiatry 79, 130 (February, 2008).
J. D. Singer, J. B. Willett, Applied longitudinal data analysis: modeling change and event occurrence. (Oxford University Press, 2003).
D. L. Collins, P. Neelin, T. M. Peters, A. C. Evans, J Comput Assist Tomogr 18, 192 (March-April, 1994).
J. D. Schmahmann, J. Doyon, A. W. Toga, M. Petrides, A. C. Evans, MRI atlas of the human cerebellum. (Academic Press, San Diego, 2000).
Asanuma K, Tang C, Ma Y, Dhawan V, Mattis P, Edwards C, et al. Network modulation in the treatment of Parkinson's disease. Brain. 2006 October; 129(Pt 10):2667-78.
Carbon M, Argyelan M, Habeck C, Ghilardi M, Fitzpatrick T, Dhawan V, et al. Increased sensorimotor network activity in DYT1 dystonia: a functional imaging study. Brain. 2010 MAR 2010; 133:690-700.
de la Fuente-Fernandez R, Ruth T J, Sossi V, Schulzer M, Calne D B, Stoessl A J. Expectation and dopamine release: mechanism of the placebo effect in Parkinson's disease. Science. 2001 Aug. 10; 293(5532):1164-6.
Efron B, Tibshirani R. An introduction to the bootstrap. New York: Chapman & Hall; 1993.
Goetz C G, Wuu J, McDermott M P, Adler C H, Fahn S, Freed C R, et al. Placebo response in Parkinson's disease: comparisons among 11 trials covering medical and surgical interventions. Mov Disord. 2008 April; 23(5):690-9.
Habeck C, Krakauer J W, Ghez C, Sackeim H A, Eidelberg D, Stern Y, et al. A new approach to spatial covariance modeling of functional brain imaging data: ordinal trend analysis. Neural Comput. 2005 July; 17(7):1602-45.
Habeck C, Krakauer J W, Ghez C, Sackeim H A, Eidelberg D, Stern Y, et al. A new approach to spatial covariance modeling of functional brain imaging data: ordinal trend analysis. Neural Comput. 2005 July; 17(7):1602-45.
Habeck C, Stern Y. Neural network approaches and their reproducibility in the study of verbal working memory and Alzheimer's disease. Clin Neurosci Res. 2007 November; 6(6):381-90.
Habeck C G. Basics of multivariate analysis in neuroimaging data. J Vis Exp. 2010(41).
Hirano S, Asanuma K, Ma Y, Tang C, Feigin A, Dhawan V, et al. Dissociation of metabolic and neurovascular responses to levodopa in the treatment of Parkinson's disease. J Neurosci. 2008 Apr. 16; 28(16):4201-9.
Huang C, Tang C, Feigin A, Lesser M, Ma Y, Pourfar M, et al. Changes in network activity with the progression of Parkinson's disease. Brain. 2007 July; 130(Pt 7):1834-46.
Katsnelson A. Experimental therapies for Parkinson's disease: Why fake it? Nature. 2011 August; 476(7359): 142-4.
Kim S Y, Frank S, Holloway R, Zimmerman C, Wilson R, Kieburtz K. Science and ethics of sham surgery: a survey of Parkinson disease clinical researchers. Arch Neurol. 2005 September; 62(9):1357-60.
LeWitt P A, Rezai A R, Leehey M A, Ojemann S G, Flaherty A W, Eskandar E N, et al. AAV2-GAD gene therapy for advanced Parkinson's disease: a double-blind, sham-surgery controlled, randomised trial. Lancet Neurol. 2011 April; 10(4):309-19.
Lidstone S C, Schulzer M, Dinelle K, Mak E, Sossi V, Ruth T J, et al. Effects of expectation on placebo-induced dopamine release in Parkinson disease. Arch Gen Psychiatry. 2010 August; 67(8): 857-65.
Ma Y, Eidelberg D. Functional imaging of cerebral blood flow and glucose metabolism in Parkinson's disease and Huntington's disease. Mol Imaging Biol. 2007 2007 July-August; 9(4):223-33.
Ma Y, Tang C, Spetsieris P G, Dhawan V, Eidelberg D. Abnormal metabolic network activity in Parkinson's disease: test-retest reproducibility. J Cereb Blood Flow Metab. 2007 March; 27(3):597-605.
Mattis P J, Tang C C, Ma Y, Dhawan V, Eidelberg D. Network correlates of the cognitive response to levodopa in Parkinson disease. Neurology. 2011 August; 77(9):858-65.
Mayberg H S, Silva J A, Brannan S K, Tekell J L, Mahurin R K, McGinnis S, et al. The functional neuroanatomy of the placebo effect. Am J Psychiatry. 2002 May; 159(5):728-37.
McClullagh P, Nelder J A. Generalized Linear Models, 2nd Edition. New York: Chapman and Hall; 1989.
Mure H, Hirano S, Tang C C, Isaias I U, Antonini A, Ma Y, et al. Parkinson's disease tremor-related metabolic network: characterization, progression, and treatment effects. Neuroimage. 2011 January; 54(2):1244-53.
Petrovic P, Kalso E, Petersson K M, Ingvar M. Placebo and opioid analgesia—imaging a shared neuronal network. Science. 2002 March; 295(5560):1737-40.
Spetsieris PG, Eidelberg D. Scaled subprofile modeling of resting state imaging data in Parkinson's disease: methodological issues. Neuroimage. 2011 February; 54(4):2899-914.
Strafella A P, Ko J H, Monchi O. Therapeutic application of transcranial magnetic stimulation in Parkinson's disease: The contribution of expectation. Neuroimage. 2006 July 15; 31(4):1666-72.
Wager T D, Rilling J K, Smith E E, Sokolik A, Casey K L, Davidson R J, et al. Placebo-induced changes in FMRI in the anticipation and experience of pain. Science. 2004 February; 303(5661):1162-7.

1. A method for identifying a pattern of brain activity associated with a placebo effect response to a placebo treatment for a disease or disorder comprising:
determining, by positron emission tomography (PET) or magnetic resonance imaging (MRI) in a subject receiving, or who has received, the placebo treatment for the disease or disorder, functional activity at each of a plurality of coordinates of the subject's brain during at least two different time points and identifying, through spatial co-variance analysis of the functional activity, which coordinates show a consistent trend over the at least two different time points in functional activity correlating with a placebo effect response to the placebo treatment,
so as to thereby determine a pattern of brain activity associated with a placebo effect response to a placebo treatment for a disease or disorder.

2. The method of claim 1, wherein the disease or disorder is a neurological disease or disorder.

3-5. (canceled)

6. The method of claim 1, wherein an improvement in the disease or disorder is determined by the subject exhibiting an improvement in at least one symptom of the disease or disorder or an improvement in at least one measurable parameter associated with the disease or disorder.

7. The method of claim 1, wherein the efficacious treatment for the disease or disorder improves at least one symptom of the disease or disorder or one measurable physical parameter associated with the disease or disorder.

8. The method of claim 1, wherein the functional activities are, or have been, determined as showing a consistent trend over at least three different time points.

9. The method of claim 1, wherein the consistent trend is a monotonic ordinal trend.

10. (canceled)

11. The method of claim 1, wherein the coordinates are three-dimensional coordinates.

12-16. (canceled)

17. The method of claim 1, further comprising determining the efficacy of a test treatment for the disease or disorder on one or more subjects by assessing if an improvement occurs in one or more symptoms of, or measurable parameter of, the disease or disorder during or subsequent to administration of the test treatment to the subject, wherein an improvement in not exhibiting the pattern of brain activity associated with a placebo effect can be attributed to the test treatment.

18. (canceled)

19. A method for determining efficacy of a candidate treatment, administered to a subject having a brain disorder, on a rate of progression of the brain disorder comprising:
a) determining, by positron emission tomography (PET) or magnetic resonance imaging (MRI) during administration of or after administration of the candidate treatment to the subject, functional activity at each of a plurality of predetermined coordinates of the subject's brain so as to determine a first pattern of activity, which coordinates have previously been identified through spatial co-variance analysis of functional activity as determined by PET or MRI in the brain of the subject or in the brain(s) of one or more other subjects suffering from the brain disorder during at least two different time points while the subject was, or subjects were, exhibiting the symptom or brain disorder as showing a consistent trend in functional activity which correlates with worsening of the brain disorder; and
b) comparing the first pattern of activity determined in step a) with a previously determined baseline pattern of activity,
wherein an expression of the first pattern of activity lower than the previously determined baseline pattern of activity indicates that the candidate treatment is efficacious in reducing the rate of progression of the brain disorder, and wherein an expression of the first pattern of activity higher than the previously determined baseline pattern of activity indicates that the candidate treatment is not efficacious in reducing the rate of progression of the brain disorder.

20. The method of claim 19, wherein the baseline pattern of activity is determined through identifying a plurality of coordinates through spatial co-variance analysis of functional activity, as quantified by PET or MRI in the brain of the subject or in the brain(s) of one or more other subjects suffering from the brain disorder during at least two different time points while the subject was, or subjects were, exhibiting the symptom or brain disorder, which coordinates show a consistent trend in functional activity which correlates with worsening of the brain disorder.

21. (canceled)

22. A method for identifying a pattern of brain activity specifically associated with a symptom of a brain disorder comprising:
determining, by positron emission tomography (PET) or functional magnetic resonance imaging (MRI) in a subject exhibiting the symptom, functional activity at each of a plurality of coordinates of the subject's brain during at least two different time points and identifying, through spatial co-variance analysis of the functional activity, which coordinates show a consistent trend over the at least two different time points in functional activity correlating with the symptom,
so as to determine a baseline pattern of activity specifically associated with the symptom of the brain disorder.

23. (canceled)

24. A method for identifying a pattern of brain activity specifically associated with efficacious treatment of a symptom of a brain disorder comprising:
determining, by positron emission tomography (PET) or magnetic resonance imaging (MRI) in a subject exhibiting the symptom and being treated with a treatment efficacious for that symptom, functional activity at each of a plurality of coordinates of the subject's brain during at least two different time points and identifying, through spatial co-variance analysis of the functional activity, which coordinates show a consistent trend over the at least two different time points in functional activity correlating with efficacious treatment of the symptom, so as to thereby determine a baseline pattern of activity specifically associated with efficacious treatment of the symptom of the brain disorder.

25. A method for identifying a pattern of brain activity specifically associated with a pre-phenoconversion rate change of a neurological disease comprising:
determining, by positron emission tomography (PET) or magnetic resonance imaging (MRI) in a subject in a pre-phenoconversion state, functional activity at each of a plurality of coordinates of the subject's brain during at least two different time points and identifying, through spatial co-variance analysis of the functional activity, which coordinates show a consistent trend in functional activity over the at least two different time points correlating with the pre-phenoconversion rate,
so as to thereby determine a pattern of brain activity specifically associated with the pre-phenoconversion rate change of the neurological disease.

26-58. (canceled)

59. The method of claim 1, wherein the subject is a mammal.

60. (canceled)

61. The method of claim 59, wherein is a mammal is a human.

62. (canceled)

63. (canceled)

64. A computer-readable medium comprising instructions stored thereon which, when executed by a data processing apparatus, causes the data processing apparatus to perform a method of claim 1.

65. A system comprising:
one or more data processing apparatus; and
a computer-readable medium coupled to the one or more data processing apparatus having instructions stored thereon which, when executed by the one or more data processing apparatus, cause one or more data processing apparatus to perform a method of claim 1.
